(19) United States
(12) Patent Application Publication
(10) Pub. No.: US 2024/0240133 A1
(43) Pub. Date: Jul. 18, 2024

(54) OPTIMIZED INDUSTRIAL BIOREACTOR AND METHOD THEREOF, WITH MUTUALLY DEPENDENT, COUPLED PROCESS CONTROL LOOPS

(71) Applicant: Bühler AG, Uzwil (CH)

(72) Inventor: Leandro BUCHMANN, Winterthur (CH)

(73) Assignee: Bühler AG, Uzwil (CH)

(21) Appl. No.: 18/562,861

(22) PCT Filed: May 25, 2022
(86) PCT No.: PCT/EP2022/064285

§ 371 Date: Nov. 21, 2023

(30) Foreign Application Priority Data
May 27, 2021 (CH) 00600/21
Jun. 15, 2021 (EP) 21179569.5

(51) Int. Cl. C12M 1/36; C12M 1/12; C12M 1/34

(52) U.S. Cl. CPC C12M 41/48; C12M 25/04; C12M 41/26; C12M 41/30; C12M 41/46

(57) ABSTRACT

The invention relates to an industrial bioreactor with a dual cycle-controlled optimization process providing an optimized cultivation process for cell cultures, cell components or metabolic products of the cells in a nutrient medium. The bioreactor comprises a reactor vessel providing controlled bioreactor conditions for the cultivation process, a control unit connected to sensory devices measuring sensory parameter values comprising at least measures related to the composition of the nutrient medium and/or concentration of the nutrient medium and/or oxygen and/or temperature and/or pH-value and/or sterility, and transmitting them to the control unit. The control unit controls and automatically optimizes the operational parameter of the cultivation process and operational parameters of a treatment process applied to the cells during the cultivation process by adjusting operational parameters of the bioreactor affecting the measured sensory parameter values.

### FIELD OF THE INVENTION

The present invention relates generally to the industrial production of biomass, and in particular to industrial processes for generating biomass by providing a controlled cultivation process for cell cultures, components of cells or metabolic products of the cell cultures using bioreactors. Even more particular, the present invention relates to industrial processes for generating biomass by cell cultivation and treatment within bioreactors e.g. by applying a pulse-regulated electric field potential.

### BACKGROUND OF THE INVENTION

### (i) Bioreactors

Bioreactors (in the context of microbial or enzymatic conversion of organic substances into acid, gases or alcohol also referred to as fermenters), typically are containers in which certain microorganisms, cells or small plants are cultivated (fermented) under applied, technically controlled conditions. The operation of a bioreactor is thus an application of the field of biotechnology that uses or harnesses biological processes (bioconversion, biocatalysis) in technical facilities. Important factors, characteristics and/or operating parameters that are controllable or steerable in most bioreactors, are, for example, the composition of the nutrient medium (also nutrient solution or substrate), oxygen supply, temperature, pH value, sterility and others. The purpose of cultivation in a bioreactor can be the controlled recovery of the cells or components of the cells or the recovery of metabolic products or the controlled metabolism of cell cultures as such. The cells, components of cells or the metabolic products can be used, for example, as active ingredients in the pharmaceutical industry, as basic chemicals in the chemical industry, or in the context of cell cultivation, for example, in the cultured food production, as e.g. cultured meat production or fermented food, e.g. tempeh, miso, koji, and soy sauce etc., or in medical applications in organ or tissue replacement treatments etc. The degradation of chemical compounds can also take place in bioreactors, as in the treatment of wastewater in sewage treatment plants. Further, the production of beer, wine and other products also takes place in bioreactors (fermenters). Today, a wide variety of organisms are cultivated in bioreactors for different purposes. Several reactor variants are available in different designs. Examples are stirred tank reactors made of metal, which can have a volume of a few to thousands of liters and are filled with nutrient solution. However, widely differing variants, such as fixed-bed reactors, photobioreactors etc. are also used.

The main purpose of bioreactors is to deliver the highest possible product yields. This is achieved in particular by creating optimal conditions for the organism or cells grown in the specific case. The conditions in the bioreactor are adapted to various parameters that prevail in the natural habitat, the cultivated organisms, or the natural environment of the cultivated cells. Typically, cultivation process parameters as the type and concentration of nutrients, temperature, oxygen content, pH value, etc. are important or critical to achieve optimized cultivation. The control of the parameters is realized using technical devices as e.g. sensors, actuators or other devices necessary to ensure a homogeneous adjustment of these parameters over the reactor chamber. In addition to the requirements of the organisms or cells, other technical, organizational, and economic factors often are to be considered that influence the choice of operating parameters. Examples can be, inter alia, the prevention of foam formation and the choice of either a continuous or a batch mode operation. Using probes or sensors, many of these parameters can e.g. be measured directly in the nutrient medium or in the exhaust air. In addition, the course of the process can usually also be assessed or monitored via these parameters. The cell density can be determined by measuring the absorbance (optical density), which in turn allows conclusions to be drawn about the product quantity. An alternative is often the measurement of the concentration of a characteristic chemical compound, e.g. the increase in concentration of a metabolic product or the decrease in substrate concentration, for example, by optical sensors or other appropriate sensors. At the beginning of a cultivation or fermentation process, typically a small amount of the microorganisms or cells to be cultivated and obtained from a pre-cultivation are added to the culture medium. This amount is called inoculum, and the process is often referred to as inoculation. The suspension obtained from the cultivation process is prepared in several process steps during so-called downstream processing.

The nutrient medium must provide the organisms/cells with all the nutrients they need for growth. These include e.g. main nutrients (macronutrients) required in larger quantities, such as carbon, nitrogen and phosphorus. Further, various trace elements (micronutrients) can be needed. Depending on the organisms/cells to be cultivated, other compounds can be required that cannot be synthesized by the organisms/cells itself (e.g. vitamins, essential amino acids, etc.). Energy-providing compounds, such as sugar glucose, can also be necessary (except in phototrophic organisms). Organisms and cells have typically an optimum temperature at which they grow and reproduce best, respectively. Exceeding this temperature can lead to irreversible damage through denaturation of the proteins, falling below it can lead to lower metabolic rates and thus to longer process durations. Temperature control can e.g. be realized by the control of applied heating and cooling circuits. When the bioreactor is started, the entire reactor contents are heated or warmed to operating temperature. In some cases, the cultivated organisms or cells generate so much waste heat through their metabolism that a cooling circuit needs to be active above a certain cell concentration. A heat exchanger can be integrated into this circuit, or the energy-bearing medium can be fed in directly. In this case, the only heat exchange surfaces available to the reaction chamber are usually the double vessel wall, or in other cases also built-in cooling registers.

Depending on the organisms or cells cultivated and/or the product, cultivation (fermentation) processes can be carried out aerobically (in an oxygen-containing atmosphere) or anaerobically (without oxygen). Since oxygen is poorly soluble in water, it can be difficult to adequately supply aerobic approaches. For example, the oxygen solubility in a fermentation medium with a temperature of 37° C. is 3-5 mg/L. The oxygen partial pressure in the reactor can be regulated by different methods, as e.g. (i) changing the gas flow rate, (ii) changing the stirrer speed, (iii) changing the stirrer tool geometry, (iv) changing the gas mixture composition, or (v) changing the head pressure (however, this also increases the solubility of other gases, for example carbon dioxide). In the case of anaerobic organisms, on the other hand, oxygen supply typically should be avoided as it can be toxic. In anaerobic approaches with anaerobic organisms, oxygen supply can e.g. enable undesired aerobic reactions, which can reduce the process yield. The cultured organisms or cells usually have a limited pH-value tolerance range with a pH optimum. The pH can e.g. be controlled with pumps coupled or linked to a pH-sensor via a control steering and/or a Programmable Logic Controller (PLC), where the pump pumps, if required e.g. phosphoric acid (H₃PO₄), hydrochloric acid (HCl), or sodium hydroxide solution (NaOH) etc., into the bioreactor to decrease or increase the pH-value. In certain cases, a defined pH-value can also be achieved by adapting the rate of feeding with substrate. To reach a defined homogenization, bioreactors can further comprise stirring devices, such as an agitator or gas injection, through which the medium is circulated. This is used to ensure a possibly homogeneous setting of various parameters throughout the reactor and thus a more uniform process flow. The mode of operation of a bioreactor can typically be distinguished between: (i) batch operation: filling of the reactor, no addition or removal during the cultivation/fermentation process, simple control, contamination unlikely; (ii) fed-batch operation: similar to batch operation; however, e.g. substrate addition during the process, as an initially high substrate concentration can be inhibiting; and (iii) continuous operation in chemostat bioreactor: uninterrupted operation by substrate addition and product removal, complex control, contamination handling, and expensive and complex downstream processes can also be carried out continuously, and thus more optimally utilized.

The operating parameters to be maintained can be strongly depending on the organism or cell type for technical or other reasons. Therefore, for particular uses, an appropriate bioreactor must often be designed, or specific types of bioreactors may be used in which the various parameters can be controlled within a wide parameter value range or window so that the reactor can be used for cultivation of different organisms or cells. A common type is the gasable stirred tank reactor in different variants (material, size, etc.). In the prior art, a classifying distinction can be made according to the type of reactor design comprising e.g.: (i) Stirred tank reactor: widespread type; the liquid phase is circulated by an agitator, gassing is performed as required; (ii) fixed-bed reactor: the reactor is filled with a solid, porous matrix on which the organisms (or enzymes) can be immobilized; the organisms thus remain in the reactor instead of being flushed out with the medium, so that the growth of the organisms is a less limiting factor (or the enzyme requirement is reduced); (iii) trickling stream reactor (trickling filter): a fixed bed is sprinkled with liquid (e.g. wastewater to be treated); the degrading organisms are located at the surface; the air (oxygen) required for degradation is introduced in countercurrent flow; (iv) photobioreactor (algae reactor, hydrogen bioreactor): for the cultivation of organisms (algae, plant (cells)) that carry out photosynthesis; the reactor is made of glass so that the necessary light reaches the organisms; the utilization of light can e.g. be optimized by plate- or tube-shaped reactors; (v) tubular reactor: in tubular reactors a plug flow can be created; and (vi) membrane bioreactor: a reactor in which (depending on the application) the reaction product, biomass or purified water can be permanently separated by membranes. Applications for this include wastewater treatment (Membrane BioReactor (MBR)), lactic acid recovery, and pharmaceutical products. Finally, the processes in bioreactors can technically be simulated e.g. by parameter processing by means of implemented reaction kinetics, although the simulation must take into account the specifics of biological processes (e.g., Michaelis-Menten theory, Monod kinetics, enzyme kinetics, enzyme inhibition, etc.).

In industrial processes and device settings, as e.g. bioreactors, control systems are used to monitor, control, steer and signal the bioreactors, facilities, plants or other equipment, and operation/processes of industrial or chemical/biochemical processes, as the cultivation or treatment processes within the bioreactors. Typically, the system that performs the controlling and monitoring uses field devices distributed at key locations in the industrial process, e.g. within the bioreactor, coupled to control circuitry by a process control loop. The term “field device” refers to any device that performs a 20 function in a distributed control or process monitoring system, including all devices used in the measurement, as e.g. sensors and measuring devices, control, monitoring and signaling of industrial processes and processing facilities. Each field device can e.g. comprise communication means, and circuitry used for communicating, in particular wired or wireless communication, with a process controller, other field devices, or other circuitry, over the process control loop. In some installations, the process control loop is also used to deliver a regulated current and/or voltage to the field device for powering the field device. The process control loop also carries data, either in an analog or digital format. Typically, field devices are used to sense or control process variables in an industrial process and/or in specific installations, if required, to monitor the local environment of the field device.

In summary, bioreactors, sensors, and methods for measuring relevant parameters are known (see for example also EP 2725095 B1, WO 2016092281 A1, CN 105044038 A). Further, automated systems of bioreactors are known where parameters and values of different nature are periodically taken as a basis for adapting the process in the successive steps (see for example also CN 105543085 A, U.S. Ser. No. 10/751,715 B1, U.S. Ser. No. 10/336,978 B2, EP 3819367 A1). However, further improvements are desirable, especially for applications in cell culture and/or microbiology controlling and steering the performance of the cultivation process.

### (ii) Cell Metabolism and Lipid Bilayer Membranes

In the light of the above-discussed, it is clear, that understanding the cell characteristics and the cell mechanisms interacting physically with the surrounding area (extracellular medium) and the area's environmental conditions is fundamental for bioreactors providing an industrial and scalable process e.g. in order to generate biomass by cell cultivation and treatment. Basis of cell reproduction is metabolism which can be defined as the set of life-sustaining chemical reactions in organisms or cells. The three core elements of metabolism are: (i) conversion of nutrient to energy to run cellular processes; (ii) conversion of nutrient to building blocks for proteins, lipids, nucleic acids, and some carbohydrates; and (iii) elimination of metabolic waste products. These enzyme-catalyzed reactions allow organisms to grow and cells to reproduce, maintain the cell structures, and respond to their environments. The word metabolism can also refer to the sum of all chemical reactions that occur in living organisms or cells, including the transport of substances into and between different cells, in which case the above described set of reactions within the cells is called intermediary or intermediate metabolism.

In particular for the intermediary or intermediate metabolism, lipid membranes serve as effective barriers allowing cells to maintain internal composition differing from that of the extracellular medium. Membrane permeation, both natural and artificial, can take place via appearance of transversal pores. For plasma membranes, lipid bilayer constitutes a major structural component. Said lipid bilayer (or phospholipid bilayer) is a thin polar membrane made of two layers of lipid molecules. These membranes are flat sheets that form a continuous barrier around all cells. The cell membranes of almost all organisms and various viruses are made of a lipid bilayer, as are the nuclear membrane surrounding the cell nucleus, and membranes of the membrane-bound organelles in the cell. The lipid bilayer is the barrier that keeps ions, proteins and other molecules where they are needed and prevents them from diffusing into areas where they should not be. Lipid bilayers are ideally suited to this role, even though they are only a few nanometers in width, because they are impermeable to most water-soluble (hydrophilic) molecules. Bilayers are particularly impermeable to ions, which allows cells to regulate salt concentrations and pH by transporting ions across their membranes using proteins called ion pumps. The amphiphilic nature of the lipid molecules, i.e. molecules possessing both hydrophilic (water-loving, polar) and lipophilic (fat-loving) properties, thus containing both polar and hydrophobic parts, determines low permeability of lipid bilayers for broad range of substances and thus allows the membranes to perform barrier function effectively in the cells. Artificial permeabilization of plasma membranes is used for various bioengineering and medical purposes. There are two alternative mechanisms of penetration through the membranes: small individual molecules can cross membranes, presumably through local defects of lipid packaging, and water-filled pores through the entire membrane can be formed, enabling non-specific transfer of various polar substances. The present invention mostly relies on the mechanisms of formation of transverse pores in lipid bilayer membranes.

Thus, understanding of the barrier properties of biological membranes is fundamental for controlling metabolism in the bioreactor. In the prior art, investigable model systems are developed, such as artificial liposomes or vesicles, which mimic the geometry and size of the cell membrane but are devoid of ion channels and the multitude of other embedded components, commonly present in the cell membrane. The simplest model of a cell membrane patch is a planar lipid bilayer. The chemical composition of the planar lipid bilayer can be chosen in advance and is therefore well defined. However, to mimic a non-curved fragment of the cell membrane, the planar lipid bilayer should separate two electrolytes. Therefore, the planar lipid bilayer is usually vertically formed across a small aperture in a hydrophobic partition that separates two compartments filled with electrolytes. Visual observation of the planar lipid bilayer in such an experimental system is limited. However, electrodes immersed in the electrolyte permit measurements of electric parameters of the planar lipid bilayer. As a planar lipid bilayer can be electrically considered as a non-perfect capacitor, the capacitance C of an ideal capacitor and the resistance R of a resistor in parallel both describe planar lipid bilayer's characteristic. Voltage-controlled and current-controlled methods enable observing electrical properties of the planar lipid bilayer and its structural changes that are reflected in the electrical characteristics. In the prior art, the capacitance C is considered as the most suitable measuring parameter for probing the stability and proper formation of planar lipid bilayer and consequently, C is almost always measured for each planar lipid bilayer investigated with electrical methods, even when other properties are technically in the focus. An exposure of planar lipid bilayer to external electrical stimulus and formation of transmembrane voltage alter both electrical parameters of planar lipid bilayer system, R and C. Molecular dynamics (MD) simulations have shown that small so-called water fingers are formed on both sides of the planar lipid bilayer in the presence of transmembrane voltage and create water or hydrophobic pores. The lipids adjacent to the water molecules inside the pores start reorienting their polar headgroups toward water molecules and stabilizing the pores into hydrophilic state, which allows more water and ions to enter the pores. These conductive paths reduce R of the planar lipid bilayer. At low transmembrane voltages, when pores are not created yet, a capacitance increase can be observed. An electro-strictive thinning of the planar lipid bilayer can be assumed as the mechanism responsible for this phenomenon. The capacitance of the planar lipid bilayer depends on applied voltage U according to the relation

$$C ( U ) = {C}_{0} [ 1 + α {U}^{2} ]$$

where C₀ is the planar lipid bilayer capacitance at zero transmembrane voltage, and a is the proportionality coefficient. Experiments revealed that its value is typically around 0.02 V⁻², while using numerical simulation models, it was assessed to be in the range of 0.053-0.082 V⁻². However, at higher transmembrane voltages, appearance of water and hydrophilic pores leads to reduction of the planar lipid bilayer capacitance, due to significant differences in dielectric constant values of lipid bilayer and water. A long-lasting exposure to strong electric stimulus causes irreversible damage to a planar lipid bilayer. Electrical measurements permit determination of the breakdown voltage value U_{br}.

(iii) Electroporation: Hydrophobic Pores and Hydrophobic Defects

Hydrophobic pores formed in planar lipid bilayer can e.g. be modeled by the known Abidor's model of electroporation, where water pores can be successfully captured by MD (Molecular Dynamics) simulations. Since such water pores are molecular-scale transient structures, they cannot be directly observed. However, the experimentally observed increase in the variance of membrane current, that is attributed to small current fluctuations due to membrane capacitance changes, can be related to the number of hydrophobic pores in the membrane during the phase transition. Even experimental information about conducting hydrophilic pores and their characteristics comes only from indirect measurements, such as current fluctuation measurements, voltage fluctuation measurements, or observing voltage drops. More recent, improved theories on the pore formation allow capturing continuous trajectories of pore formation, both in the absence and presence of mechanical stress. Those theories may explain the complex behavior of pores during their formation under lateral tension, even in various loading rates regimes that can be studied in giant unilamellar vesicles (GUV) aspiration experiments. Namely, at low loading rates, GUV membrane ruptured at small tensions and, according to theory, membrane breakdown is governed by pore expansion. On the other hand, at high loading rates, GUV membrane rupture occurs at high values of tensions and is theoretically limited by water pore formation, which is lipid type (curvature) dependent. Akimov's theory predicts that the planar lipid bilayer, exposed to the electric field, behaves similarly as in the presence of lateral tension. Experimentally, it can be shown that an increase in the permeability of the lipid bilayer of biological membranes, manifested in the form of discrete conductivity jumps at electrical measurements, in the leakage of the contents of liposomes and in the enhancement of flip-flop, occurs under the action of an electric field and membrane tension, close to chain melting transitions as well as at the addition of detergents. It seems likely that the molecular mechanisms for increasing the permeability of the bilayer in all of these cases have much in common. In particular, it seems plausible to associate the increase in the lipid bilayer permeability with the formation of hypothetical hydrophobic defects and hydrophilic pores. Although available experimental methods do not allow direct studies of these structures, the occurrence of current fluctuations in flat bilayers, leakage of liposomes, and flip-flop enhancement can serve as indirect evidence for the existence of hydrophilic pores in the bilayer.

To understand the formation of hydrophilic pores, it can be assumed that a hydrophilic pore is formed in an originally intact bilayer through an intermediate state referred to as hydrophobic defect. There are evidences from electroporation experiments for the existence of small, metastable “pre-pores”, referred to as hydrophobic defects, in the stage of hydrophilic pore formation process. However, the structure, characteristics and process of occurrence of hydrophobic pores as well as the molecular mechanisms for the formation of primary hydrophobic defects are still unclear, and not explained e.g. by using the methods of molecular dynamics. Hydrophobic defects can occur in an intact membrane as a result of thermal fluctuations. However, they do not lead to macroscopic consequences, since the frequency of their appearance is small. The experimentally observed increase in membrane permeability may be associated with an increase in the frequency of occurrence and the number of hydrophobic defects under the action of a stimulus (electric field, thermodynamic changes during a phase transition, etc.). For example, the occurrence of a sufficiently large number of hypothetical hydrophobic defects during a phase transition can be associated with an increase in area fluctuations. One difficulty of describing this stage of pore formation and evolution lies in the lack of experimental data associated with hydrophobic pores. At present, pre-pores (hydrophobic defects) are only indirectly detected by observing the effects of “memory” in membranes and flickering of conducting events during the lifetime of the pre-pores. Thus, experimental data associated with the existence of hydrophobic pores in the lipid bilayers and allowing to measure their number and cause of occurrence are of a fundamental interest. As a possible approach, the Smoluchowski equation with an energy profile of a special type and an assumed hydrophobic pore source term can e.g. be used to capture the process of hydrophilic pore formation in a lipid bilayer at the gel-liquid phase transition. The source term reflected the occurrence of molecule packing defects in a lipid bilayer at phase transition. That permitted to model the temporal sequences of current impulses occurring at phase transitions with various thermodynamic characteristics. In the classical theory of electroporation, the hydrophobic pores are presented in the form of water columns in contact with the hydrophobic lipid tails. Such a pore does not allow the transit of ions but can change the capacitance of the membrane by changing its dielectric permittivity. As a hydrophobic pore opens or closes under a fixed voltage regime, current fluctuations must thus be induced, with the current mean being equal to zero. For modelling the increase in current variance through membranes can be measured, the temperature of which decreases, approaching the phase transition temperature. The modelling then connects the number of hydrophobic pores with the variation of membrane current. The number of hydrophobic pores as obtained from measurements can, thus, be compared with the theoretical value as provided using the Smoluchowski equation with a source of hydrophobic pores reflecting the occurrence of packing defects in the lipid bilayer at phase transition. Based on this model, estimates of the number of hydrophobic pores in the membrane during the phase transition can be obtained.

(iv) Nanosecond Pulsed Electric Field Application (nsPEF)

The last few decades have seen an increased interest in the application of pulsed electric fields (PEF), which has been particularly applied in the medical and biotechnology sectors. They have been shown to have broad applications in the food processing industry, for cryopreservation of cell lines, enhancing gene or drug uptake by cells, and induce cell death through necrotic or apoptotic pathways. Understanding how electric fields influence cell behavior is important when considering how to technically improve or expand current treatment options. Above, we already discussed the cell and its intracellular contents, and more specifically the membranes that surround them, where electric fields are expected to interact.

In the technical field of bioengineering, electroporation (also electro-permeabilization) is defined as the exposure of biological materials (cell suspensions, tissues) to high voltage electric pulses provoking cell membranes to become more permeable to molecules that otherwise cannot cross them. Notwithstanding the technical and theoretical problems in modelling and understanding the process, electroporation is nowadays widely used as a basis for applications in biotechnology and medicine such as electrochemotherapy, gene electrotransfer, tissue ablation, extraction of various compounds, and microbial inactivation in food preservation. In the application of electroporation, it could be shown that shorter electric pulses of nanosecond duration (nsEP: nanosecond Electric Pulses; also referred as nsPEF: nanosecond Pulsed Electric Field) have more profound effects on the cell interior than longer pulses of micro- and millisecond duration, and thus nsPEF can be used as a tool for intracellular manipulation without chemical intervention. With the development of new pulse generators that produced ultrashort pulses of very high electric fields of several tens of MV/m (Megavolt/meter (electric field strength)), the effects can be demonstrated on cells, the nanosecond electric pulses affecting cell organelles, increase intracellular calcium, and provoke apoptosis and stress responses. Further, also the plasma membranes are affected. The pores produced by nsPEF are small, of nanometer scale, and are thus also referred to as “nanopores”. Cells exposed to nsPEF exhibit membrane permeability to both propidium (PI) and trypan blue (TB), classical indicators of membrane permeabilization, but detecting influx of these dyes and other small molecules after nanosecond pulse exposure requires methods with greater sensitivity than those used for longer pulses. However, using different cells, pulse parameters, exposure configuration and detection methods can lead to different results. Thus, the industrial use of nsPEF in bioreactors is not trivial and may have complex dependences the technical setup of the bioreactor and the used operational parameters.

The application of nsPEF to cells can e.g. be classified into distinct frequency regions, as: (A) 1-10 ns, (B) 11-100 ns and (C) 101-999 ns. With the use of nanosecond pulses, as pulses shorten, more intracellular effects and less effects on plasma membrane are typically expected. The first category (A) includes very short pulses, 1-10 ns, with rise times of a few ns (mostly shorter than the electrolyte relaxation time). In this regime the dielectric properties of the membrane and the intracellular and extracellular media dominate pulsed electric field effects on membranes, and the Maxwell-Wagner polarization of the membrane by migration of mobile charges is less important than it is for longer pulses. Moreover, proportionally greater effects can be expected on intracellular membranes with 1-10 ns pulses than with longer pulses. In the second category (B), the pulse duration is less than the charging time of the plasma membrane. The third category (C) includes nsPEF with durations longer than the plasma membrane charging time. It is a demand in the technical field, to provide an optimized bioreactor and bioreactor process, inter alia, controlling the role of pulse duration in regard to the accompanying measurable effects of nsPEF on biological cells, as eukaryotic human, animal, and plant cell cultivation in bioreactors. Such an optimized bioreactor and bioreactor cultivation process has to be enabled to response on various conditions, e.g. since the occurrence of changes in the plasma membrane can significantly depend on the nsPEF pulse duration, and/or the occurrence of intracellular effects can significantly depend on the nsPEF pulse duration, where PM effects may be greater with longer pulses and intracellular effects may be greater with shorter pulses.

Further for the technical setup, during the electroporation process biological cells are exposed to pulsed electric fields with specific electrical parameters, as amplitude, duration, shape, number of pulses and pulse repetition rate. The duration of the pulse can be specified as the full width at half maximum (FWHM) and a description of the pulse shape can be enhanced with rise and fall times of the pulse. In order to exactly specify the technical method and thus to enable the reproduction of the cultivation and treatment process within the bioreactor, it is typically necessary to exactly determine these electrical parameters. Some electrical parameters such as number of pulses and pulse repetition rate are relatively easy to capture. Other electrical parameters are more difficult to determine, because it is difficult to measure the time course and distribution of the nanosecond electric field within the exposure configuration during the delivery in the bioreactor. In the prior art on the electroporation of biological cells by nsPEF there is a need for the precise determination and optimized control of the electric field to which biological cells are exposed in bioreactor and the cultivation and/or treatment process.

There are several factors known influencing the outcome following membrane exposure to an electric field. Generally it can be assumed that the initial step in membrane breakdown following PEF exposure is the magnitude of the induced transmembrane potential. Thus, the electric field strength can play an important role in this process. Finding the threshold for this response has been the subject of many bioreactor systems seeking to exploit this consequence of PEF application. Achieving this threshold, however, is highly dependent on the duration of the pulse delivered, such that eliciting a response using a shorter pulse would require a stronger electric field than the same response using a longer pulse. For example, using pulses ranging from Ins to 100 ms to stimulate specific muscle cells, an applied voltage of 4.5 kV may be required for a 1.8 ns pulse to elicit a similar response to a 100 ms pulse delivered at 0.02 kV. In addition to the electric field intensity and pulse duration, other important features are; the number of pulses and the frequency at which they are delivered.

Another feature becomes evident when applying different pulse shapes. For example, using μsPEFs with the electric field intensity required to permeabilize 50% of the cells, this may be reduced by 20% when symmetrical bipolar pulses are used. The reason may lie in the fact that i) the polar asymmetry following a unipolar pulse is counterbalanced when a bipolar pulse is delivered and ii) bipolar pulses increase the probability that a non-spherical cell are permeabilized. However, in contrast to this, there may also occur a dampened effect when bipolar nanosecond pulses are used. For example, the addition of a second phase (of opposite polarity) may cancel the effect of the initial monopolar phase in both intracellular calcium mobilization and cell survival. A similar dampening effect can be observed for calcium uptake as well as measures of plasma membrane integrity when comparing bipolar effects to monopolar effects. Notwithstanding the existing models for capturing the differences between us and ns bipolar pulses, it can be stated that the duration of the pulse is critical and is based on a biological constant known as the membrane charging constant

As discussed above, the induced transmembrane potential can be assumed to be due to the accumulation of ions along the cells membrane. This process requires time, and is referred to as the charging time, or charging constant. Although it varies slightly between cells, the charging time is on the order of a few hundred nanoseconds. Pulse durations that are longer than the charging constant will have very different effects those that are shorter. As mentioned above, in this application, pulses that are longer than the charging time will be referred to as pulsed electric fields (PEF), and those that are shorter will be referred to as nanosecond pulsed electric fields (nsPEF).

At longer durations, in the micro- or millisecond range, PEF have been shown to be effective at delivering proteins, drugs, and DNA across the cell membrane, i.e. PEF makes the membranes permeable which allows molecules to diffuse into the cell. By adjusting the number, frequency and intensity of the pulses one can reversibly or irreversibly permeabilize the plasma membrane. In the prior art, irreversible electro-permeabilization (IRE) has been mainly applied as an electrical treatment for tumors. With IRE, the cell is unable to repair the damage generated by the electric field. Contrary to IRE, reversible electro-permeabilization utilizes the ability of the membrane to repair itself as an advantage. Of the two, reversible electro-permeabilization has been most thoroughly investigated, and offers a greater number of potential applications. Gene electrotransfer (GET) is one example of such an application, where DNA is introduced into the cell and makes it possible to regulate the production of a desired transgene. In the medical field, vast number of disorders that can potentially be treated using reversible electro-permeabilization systems, including Parkinson's disease, HIV/AIDS and cancer etc. Electrochemotherapy (ECT) is another technical application for reversible electro-permeabilization, where chemotherapeutics (or high concentrations of calcium) can be injected systemically or locally followed by electro-pulsation of the tissue.

In nsPEF, when pulse durations shorter than the charging constant are applied, the plasma membrane no longer shields the intracellular environment, and very different effects typically occur. Effects such as mitochondrial depolarization, caspase activation and nuclear condensation, all effects typical of cell death by apoptosis, can be observed. Additional effects such as cytoskeleton disruptions, including microtubule and actin assembly have also been observed. Whereas intracellular effects are specific to nsPEF, there is mounting evidence suggesting additional effects occur on the plasma membrane. For example, membrane permeability was found to be enhanced following nsPEF exposure. It can be assumed that these ultra-short fields may exert effects directly on transmembrane protein channels in the plasma membrane. In prior art, experimental evidence exists that supports this, where nanosecond pulses have resulted in direct activation of voltage-gated channels (cf. FIG. 2).

Disregarding the application in the medical field, the application of pulsed electric fields (PEF) and nsPEF can be used in various areas of the food industry and bioprocess engineering. Here the objective is to influence the product cell structure and/or the metabolism of the cultivated cells. The cells can be plant or animal cells, e.g. those of a potato or meat, but also of microorganisms. The cells show differences in physical characteristics, as size and particularly in their composition. However, all cells are surrounded by a membrane with phospholipids as its main component (cf. FIG. 1). Also for the bioprocess engineering, e.g. in bioreactors, due to the properties of the phospholipids, the membrane can be seen as an insulator, and the cell therefore has a natural charge, already mentioned above as trans-membrane voltage. For PEF, the application of an external voltage, a charge accumulation and an increase in the potential is induced and an electrical compression is triggered. This results in the formation of a pore in the membrane, which is the above-discussed process of pore induction with PEF (electroporation). Also in bioprocess engineering, depending on the applied pulse characteristic (electric field strength, pulse shape and width, energy input), reversible or irreversible pores can be formed. Reversible pores are hydrophilic and close spontaneously after a short time. With a higher intensity of the electric pulses and a longer treatment, the initially hydrophilic pores turn into hydrophobic pores that cannot be healed again. This causes permanent damage to the cell. For the microorganisms contained in the product, the loss of the boundary to the environment means the loss of viability. Plant cells, e.g. potato cells, loose their internal cell pressure (turgor) when subjected to the PEF treatment and the increase in the membrane permeability results in an easier mass transport, for example when extracting valuable ingredients. As such, PEF treatment applications can be used for a variety of different applications, as inactivation of microorganisms, the reduction of vegetative germs, inactivation of bacterial endospores, drying of products etc.

In addition to plant cells, also animal cells, e.g. meat cells, can be influenced by PEF or nsPEF treatment. The present invention, inter alia, relates to systems and methods for enhancing cultured meat production, such as livestock-autonomous meat production. The conceptual promises of “cultured meat” (e.g., animal-autonomous meat production by in vitro cell culture, tissue engineering, and food technology meth-ods) include increased production efficiency, reduced environmental impacts, expanded culinary application utility, enhanced nutritional value, cruelty-free production and improved food safety relative to conventionally produced meats. Technologies to date, however, have not advanced sufficiently to support scalable, economically sustainable and controllable (quality/characteristics) production. The current laboratory-scale cultivation of prototype tissues has utilized primary animal components such as animal tissues and serum, thereby largely negating the advantages of animal-autonomous meat production. Hence, current methods fail to resolve the animal dependence from cultured meat production sufficiently to realize the conceptual promises of “cultured meat” and provide a commercially advantageous product. Therefore, there is a need to provide new and improved methods for scalable meat cultivation from a self-renewing source in vitro for dietary nutrition and other applications. Further, the technical challenges in manufacturing cultured meat products, unseen the need for a production of large quantities of cells, include producing products having sensory qualities appealing to the consumers, including visible appearance, texture, flavor and aroma. However, main challenge remains to scale-up the production processes in order to manufacture the meat products in large quantities suitable for human consumption. A particularly challenging mission is the direct production of a meat portion suitable for serving, rather than separate cultured meat aggregates or layers that need to be fused or connected in order to obtain a meat portion suitable for serving.

Attempts for producing large quantities of mammalian cells, and to adhering the cells to a substance have also been taken in the pharmaceutical industry, for example for producing stem cells for therapeutic use and for tissue and organ grafting. For example the patent applications U.S. Pat. No. 6,911,201 and US 2010/0233130 disclose methods of producing undifferentiated hemopoietic stem cells using a stationary phase plug-flow bioreactor. In some embodiments, the methods comprise seeding undifferentiated hemopoietic stem cells or progenitor cells into a stationary phase plug-flow bioreactor in which a three-dimensional stromal cell culture has been pre-established on a substrate in the form of a sheet, the substrate including a non-woven fibrous matrix forming a physiologically acceptable three-dimensional network of fibers. The document WO 2008/152640 discloses a method of transplanting the three-dimensional stromal cell culture comprising hematopoietic stem cells into a recipient. Another example can be given by U.S. Pat. No. 9,127,242 disclosing a single-use, single or multiple tissue, organ, and graft bioreactor and environmental control system.

In summary, nsPEF induced growth stimulation by an electric field in bioreactors is known in the prior art, in particular there are documented effects in fungi, soy, microalgae, and other cells. However, controlled and reproducible growth stimulation was currently not possible. Electric fields and PEF may have precise stimulation windows, but controllable and reliable growth and/or compound stimulation has only been achieved under nsPEF conditions. However, since growth stimulation based on nsPEF processing yielded promising results using batch systems, nsPEF has the potential to increase growth as well as specific cellular compounds while maintaining techno-functional properties of the remaining compounds, if suitable systems are provided. Moreover, nsPEF-based growth/cellular compound stimulation has the potential to increase resource efficiency, economic viability, and the affordability of the derived products, thereby meeting the demands of a growing world population.

### SUMMARY OF THE INVENTION

It is an object of the present invention to overcome the disadvantages and technical problems known from the prior art. In particular, the aim is to provide an accurate and efficient industrial bioreactor with an automated optimization process allowing to provide a reliable, controllable, and automated detection of optimal processing parameters for an industrial cultivation process in a bioreactor, and thus an optimized steering of the cultivation process in the bioreactor. It is a further object of the invention to provide an automatically controlled and reproducible, nsPEF induced growth stimulation for cell cultures in a bioreactor, in particular for electric fields and PEF applications having dedicated stimulation windows for controllable and reliable growth and/or compound stimulation.

According to the present invention, the objects of the invention can be realized and achieved by means of the elements and combinations particularly depicted in the independent and dependent claims. It is to be noted that both, the foregoing general description and the following detailed description are exemplary and explanatory only and are not restrictive of the invention as described.

According to the present invention, the above-mentioned objects for an industrial bioreactor with a dual cycle-controlled optimization process providing an optimized cultivation process for cell cultures, cell components or metabolic products of the cells in a nutrient medium and appropriate method are achieved, particularly, in that the bioreactor comprises a reactor vessel providing controlled bioreactor conditions for the cultivation process, a control unit connected to sensory devices measuring sensory parameter values comprising at least measures related to the composition of the nutrient medium and/or concentration of the nutrient medium and/or oxygen and/or temperature and/or pH-value and/or sterility, and transmitting them to the control unit, wherein the control unit controls and/or steers the cultivation process by adjusting operational parameters of the bioreactor affecting the measured sensory parameter values, in that the dual cycle-controlled optimization process comprises a cultivation optimization cycle optimizing cultivation performance of the cultivation process by adjusting operational first parameters of the bioreactor identifying a biologically optimized treatment window, and a treatment optimization cycle optimizing a treatment process applied to the cell culture during the cultivation process within the optimized treatment window by applying operational second parameters of the bioreactor, in that the cultivation optimization cycle comprises measuring first sensory parameters by first sensory devices capturing the cultivation performance of the cultivation process, wherein the first sensory parameters are transmitted to a first analyzer reconciling between a target cultivation performance and the measured cultivation performance (i.e. triggering the measured cultivation performance for a target cultivation performance to be reached) and if the target cultivation performance is not met, the operational first parameters are adjusted and the measuring and reconciliation is reiterated (i.e. an adjustment of the operational first parameters and a further reiteration of the reconciliation is triggered), and in that if the target cultivation performance is met, the treatment optimization cycle is triggered comprises measuring second sensory parameters by second sensory devices capturing the treatment performance of the treatment process by measuring the treatment induced deviation in the cultivation performance, wherein the second sensory parameters are transmitted to a second analyzer reconciling between a target treatment performance and the measured treatment performance and if the target treatment performance is not met, the operational second parameters are adjusted and the measuring and reconciliation is reiterated, otherwise the dual cycle-controlled optimization process is completed. One of the advantages of the invention is, inter alia, that the inventive industrial bioreactor with a dual cycle-controlled optimization process allows for the first time to provide a reliable and controllable detection of optimal processing parameters for an industrial cultivation process in a bioreactor. This is achieved by detecting in a first step the optimal biological treatment window based on the cell characteristics of the cells to be cultivated. In a second step, the technical treatment is optimized, i.e. the operational parameters of the treatment to be applied. This first step can continue during the whole treatment and, thus, can serve to identify the treatment stop while the second step allows to quantify the process efficiency and thus detects the percentage increase of the cultivation/growth efficiency and thus further also allows the prediction of the process runtime.

As an embodiment variant, the control unit can e.g. comprise a machine-learning or artificial-intelligence based module capturing the measured treatment performance measured by means of dielectric spectroscopy system and/or the measured treatment performance measured by means of the flow cytometer, wherein the operational first parameters and/or operational second parameters are automatically adapted by the control unit, wherein at least the first and secondary sensory parameter are applied as input values to the machine-learning or artificial-intelligence based module, and wherein the output of the machine-learning or artificial-intelligence based module triggers the adjustment of the operational first parameters and/or operational second parameters until the target cultivation performance with the applied treatment process is reached. For example, at least some of the measured sensory parameters can serve as input to the machine-learning module, while at least some of the operational parameters are subject to the output-values of the machine-learning or artificial-intelligence based module. The machine-learning module can e.g. be based on supervised or unsupervised learning structures. As an example among other suitable machine-learning structures, the machine-learning module can e.g. convert the measured first and/or second sensory parameters into a sequence of assigned binary input codes and process the sequence of the assigned binary input codes by applying a maximum likelihood parameter estimation for the training of a multi-dimensional data structure of the machine-learning module with the variable hidden Markov model parameters, wherein the elements of the sequence of storable parameter states of the Markov chain are assumed to be independent measurements of each other and wherein the model parameters of the multi-dimensional data structure are varied by maximizing the multiplied product of the probabilities in order to obtain the trained model parameters of the multi-dimensional data structure. The model parameters of the multi-dimensional data structure can e.g. be iteratively varied until a predefined convergence threshold is reached. For determining said threshold value of a score (as e.g. given by a loss function) indicating or providing a measure for the optimization of the operational first and/or second operational parameters, an averaging process can e.g. be applied based on the different pattern of first and/or second sensory parameters of the sensory and/or measuring data of an identified time frames. In an embodiment variant, the sensitivity of the chosen operational parameters can e.g. be automatically tuned based on dynamic adjustments of the threshold value. This embodiment variant has inter alia the advantage, that the convergence speed by training the variable hidden Markov model parameters of the multi-dimensional data structure can be optimized. The embodiment variant has, inter alia, the advantage that it provides a novel method and bioreactor for automated detection, measuring and triggering of an optimized setting for the first and second operational parameters affecting the cultivation of the cell cultures and the applied treatment used in industrial bioreactor. It provides an efficient automated system for controlling and monitoring small- to large-scale industrial bioreactors (e.g. used for cultured meat production), which typically are difficult to control. In addition, this embodiment variant has, inter alia, the advantage that the AI integration allows to treat also completely unknown organisms without evaluating appropriate settings for the first and/or second operational parameters of the bioreactor empirically, since the system optimizes itself by means of the actual/target comparison.

### BRIEF DESCRIPTION OF THE DRAWINGS

The present invention will be explained in more detail, by way of example, with reference to the drawings in which:

FIG. 1 shows a diagram schematically illustrating a biological membrane. Core for understanding how cells interact with their environment is the boundary that separates the cell, i.e. its membrane. Cell membranes (FIG. 1) are composed of a series of polar lipid molecules that have hydrophobic and hydrophilic regions organized into a bilayer. This structure displays self-organizing properties in an aqueous environment that results in a boundary condition separating the cytoplasm from extracellular fluids. A very similar arrangement of amphipathic lipid molecules within the cell serves to separate organelles from the cytoplasm. These membranes are critical for selective transport into and out of the cell. In addition to the phospholipid bilayer, a host of other lipids, proteins and carbohydrates can be identified. These molecules are highly variable in their distribution along the membrane, each serving different functions ranging from signaling molecules to structural support etc. Transmembrane protein channels are of particular interest to the topic. The cell membrane, as shown in FIG. 1, is a highly complex structure whose fundamental unit is the phospholipid molecule (bottom right of FIG. 1). The amphipathic structure of this molecule results in the formation of a bilayer in aqueous solution. Embedded within the membrane, multiple additional lipids, proteins and carbohydrates serve to communicate with the external environment.

FIG. 2 shows a diagram schematically illustrating ion channels classified by the stimulus which modulates their activity. Transmembrane proteins, to a large degree, dictate the function of the cell. Some serve as signal conduits, whereby extracellular molecules interact with the protein leading to a specific intracellular effect. Others serve as selectivity filters responsible for modulating, either passively or actively, the passage of ions. These ion channels are highly variable and are critical for many cell processes. Ion channels fluctuate between open and closed states, and these states can be influenced through multiple mechanisms, as illustrated in FIG. 2. Some examples include voltage-gated channels, mechanically-gated channels, or ligand-gated channels. Upon activation, these channels become permeable to specific ions. They can be highly specific, meaning they allow just one type of ion to pass, or charge-specific allowing either cations or anions to pass. Voltage-gated channels can be activated by fluctuations in a cells transmembrane potential. Ligand-gated channels are activated through signal molecules that bind on extra- or intracellular sites of the channel. Mechanically-gated channels are modulated through changes in shape associated a physical stimulus. The selective nature of these ion channels is responsible for the generation of a transmembrane potential, which is equally present and varies among intracellular organelles as it is across the plasma membrane. The chemical basis of this voltage-gradient, known as the resting membrane potential, is the uneven distribution of ions inside and outside of the cell. The electrical properties of ions, combined with the conductive properties of protein channels and the insulating properties of the membranes lipid bilayer, have allowed the cell to be modeled as an electric circuit. This has been useful when modelling electrical communication between cells for example, but also when developing technical cultivation and/or treatment strategies to modulate cell activity and cell cultivation.

FIG. 3 shows a diagram schematically illustrating the cell working as an electrical circuit, captured by the Hodgkin-Huxley parametrization. The Hodgkin-Huxley parametrization (introduced by A. Hodgkin and A. Huxley) proposes a working structure or model to capture the ionic mechanisms responsible for the propagation of electrical impulses along a giant squid axon. As a general overview shown by FIG. 3, the Hodgkin-Huxley parametrization assumes the lipid bilayer as parallel capacitors capable of storing charge and ion channels as variable resistors capable of passing current. The transmembrane potential is generated across the cell membrane and fluctuates according to the activity of the ion channels. This model assumptions are important for capturing or modelling certain biological phenomena such as electrochemical communication between cells, particularly between electrically excitable cells in nervous and muscular tissues. Another application for this model extends into the technical field interesting for the present application, for modulating and steering cellular activities using applied electric fields in bioreactors. In FIG. 3, the lipid bilayer is represented as a pair of parallel capacitors and the transmembrane ion channels are represented as variable resistors.

FIG. 4 shows a diagram schematically illustrating the electric field interaction with the cell membrane. When cells are exposed to an electric field, an electric force is generated, which acts on ions both inside the cell and in the external media. Because ions are charged ions, this force causes them to move along the electric field lines. Whereas the extra- and intracellular solutions are conductive, the cells lipid membrane is non-conductive. As a result these ions will accumulate along the membrane and generate a large transmembrane potential. At a certain point, which is assumed in the prior art to be approximately 1V, the induced voltage exceeds the membrane capacitance and breakdown of the membrane occurs. When considering the diameter of the membrane is approximately 10 nm, the electric field strength associated with this threshold is on the order of a MV/cm. This process, which is termed electroporation or electro-permeabilization (see above) is associated with enhanced membrane permeability. To capture this effect, the Schwan parametrization respectively relation was developed, given by:

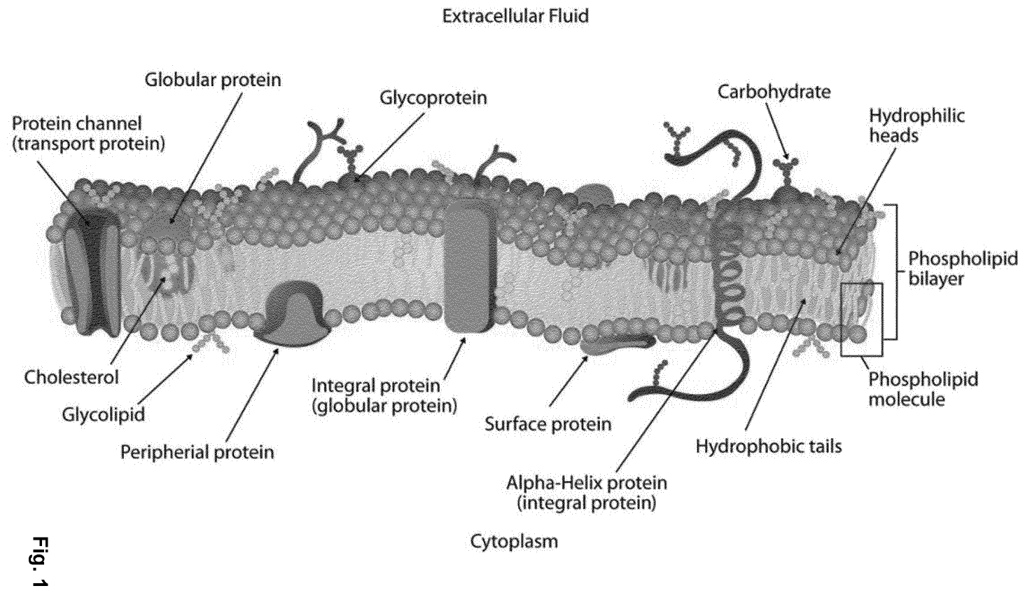

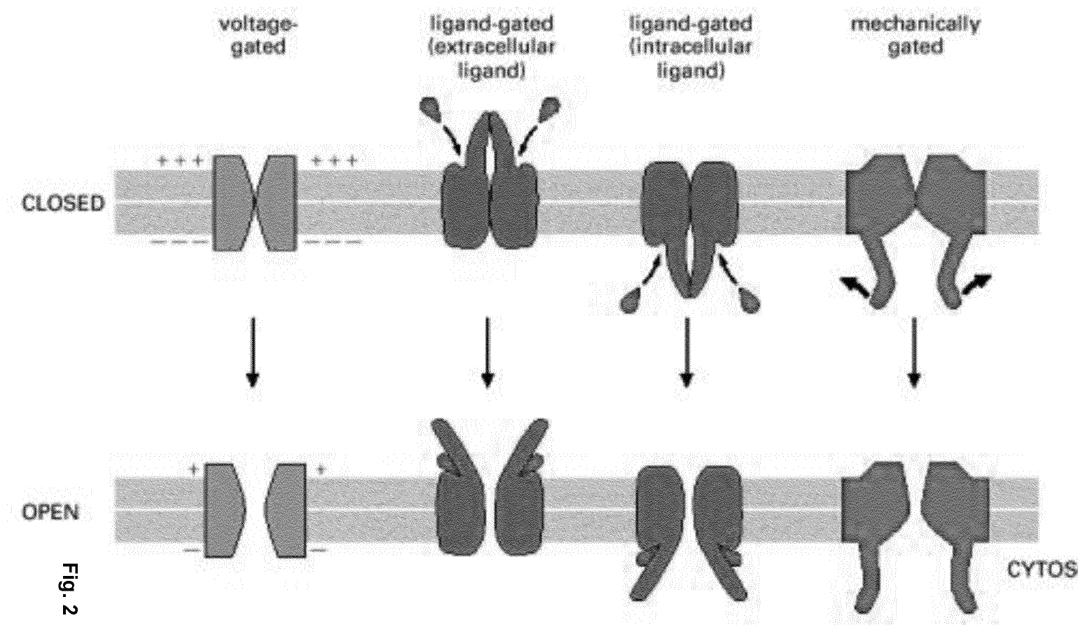

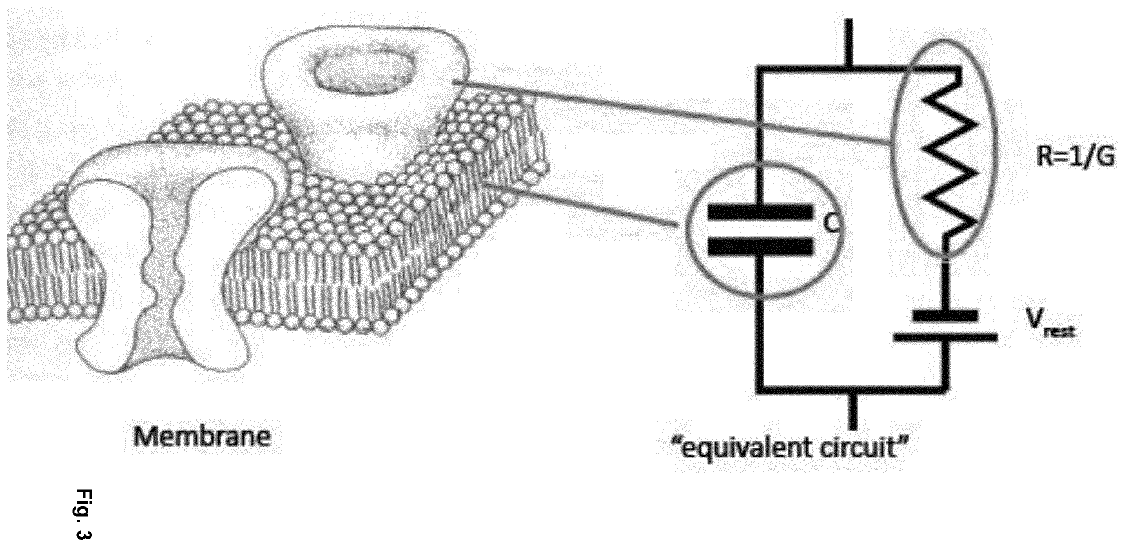

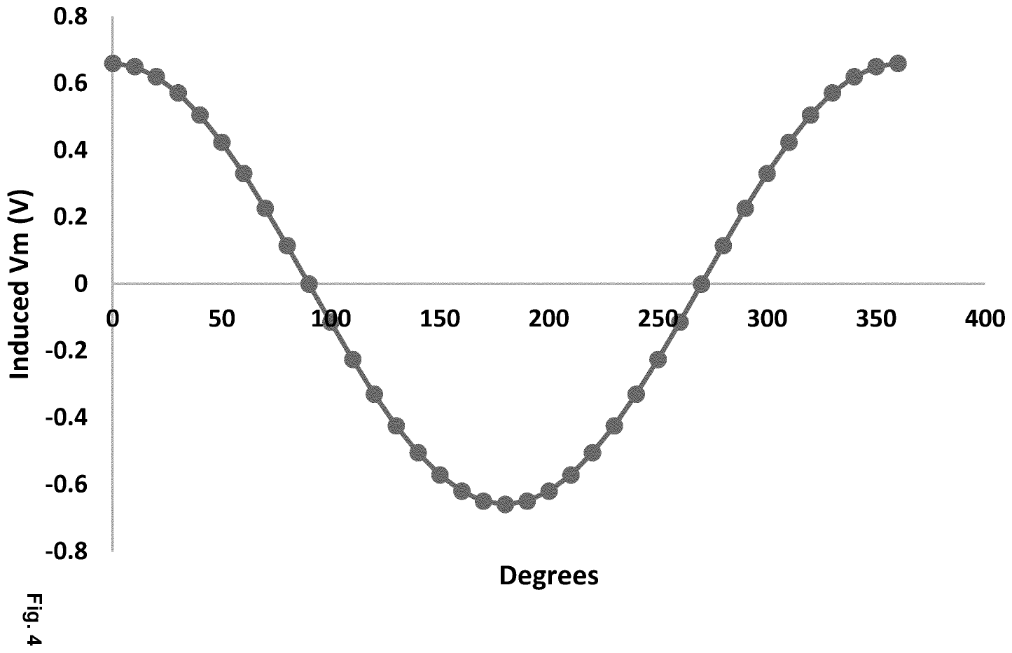

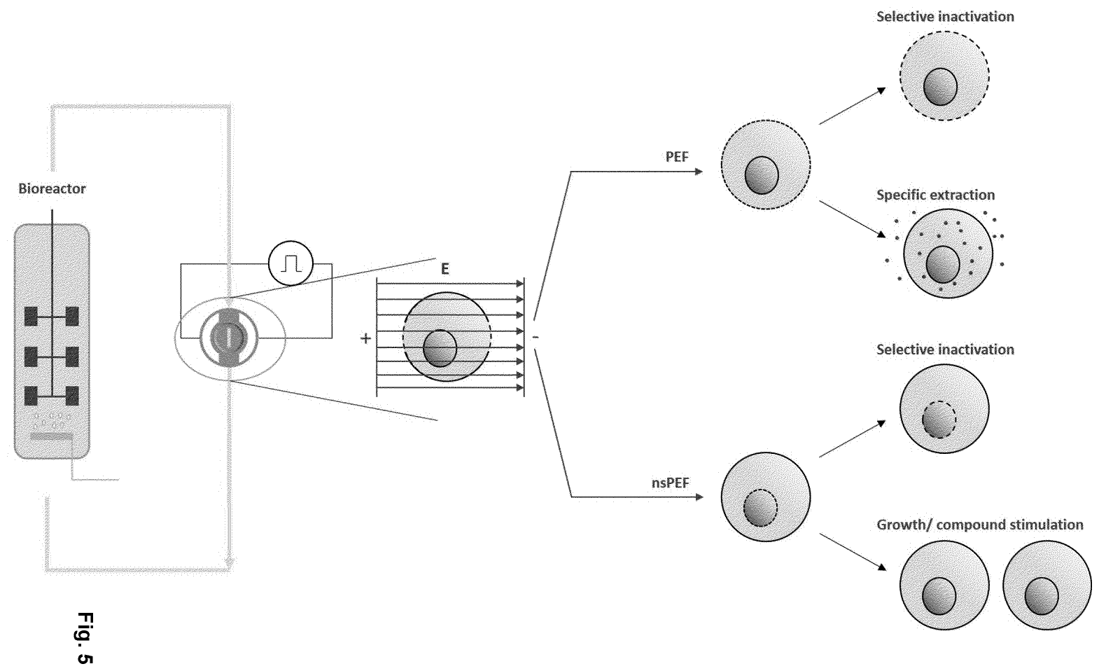

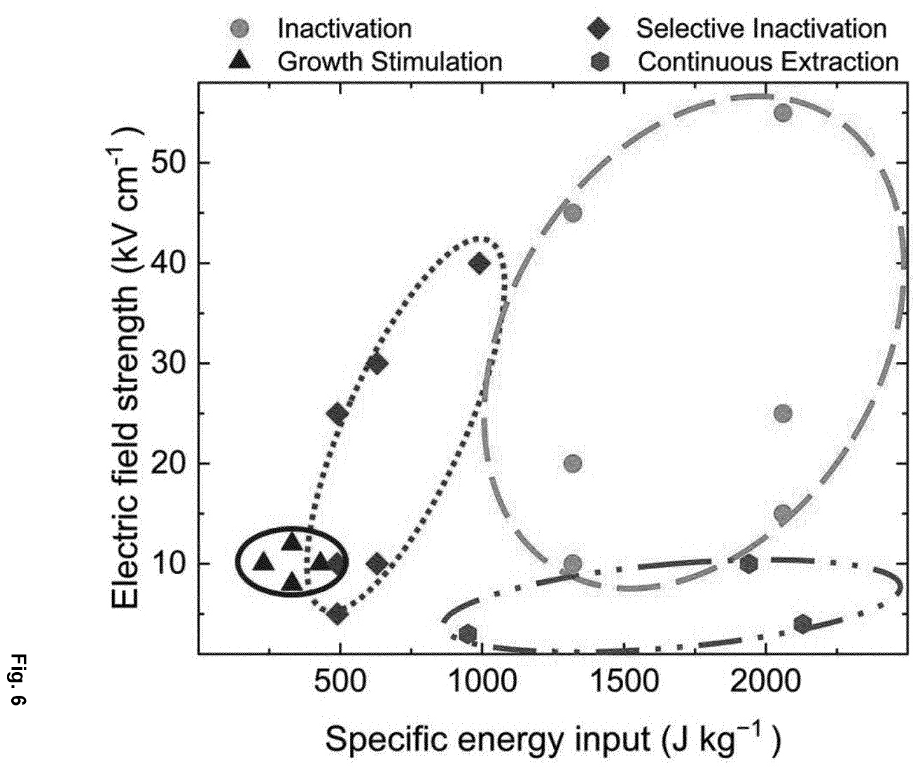

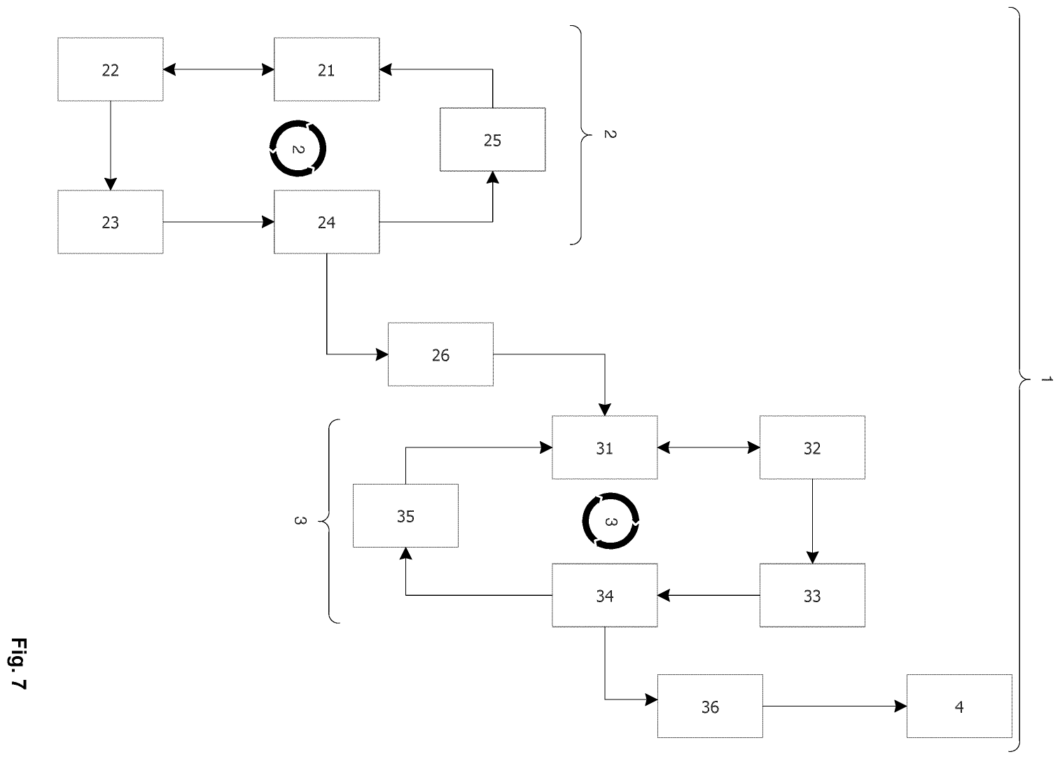

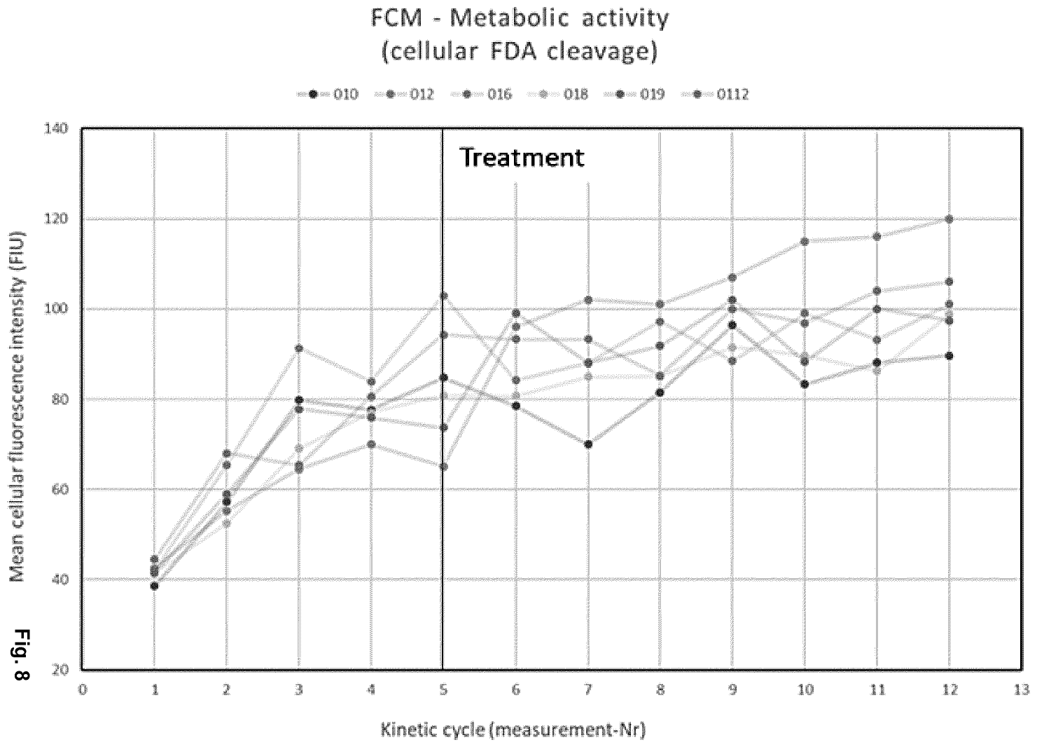

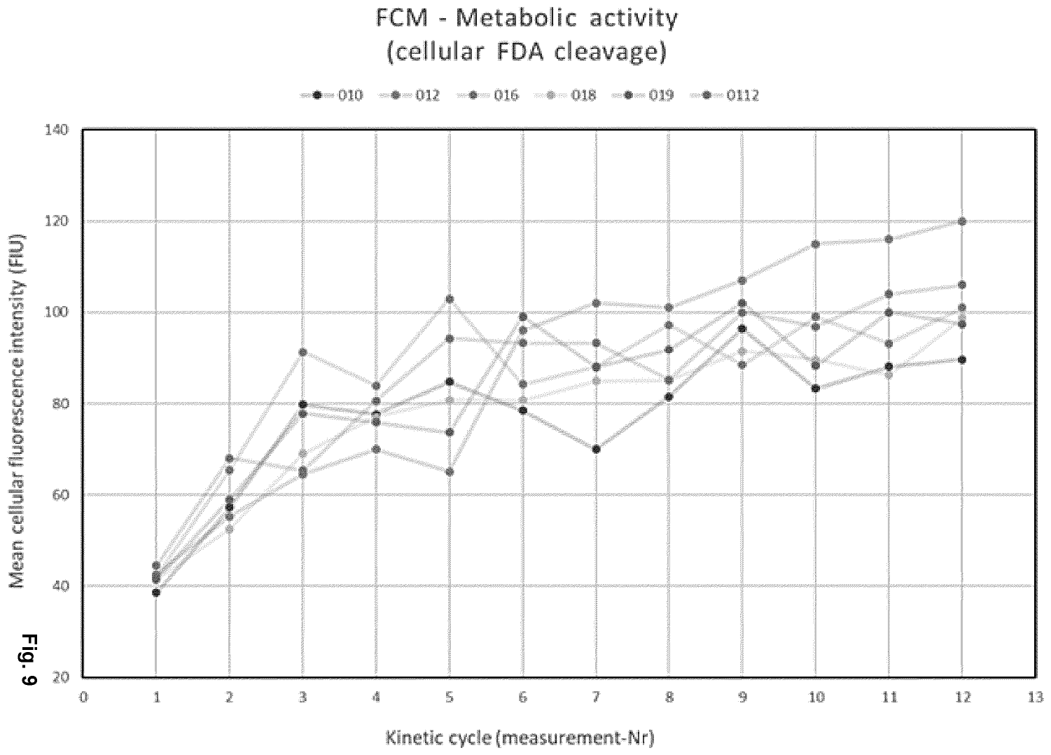

$${V}_{m} = \frac{3}{2} ER cos θ$$

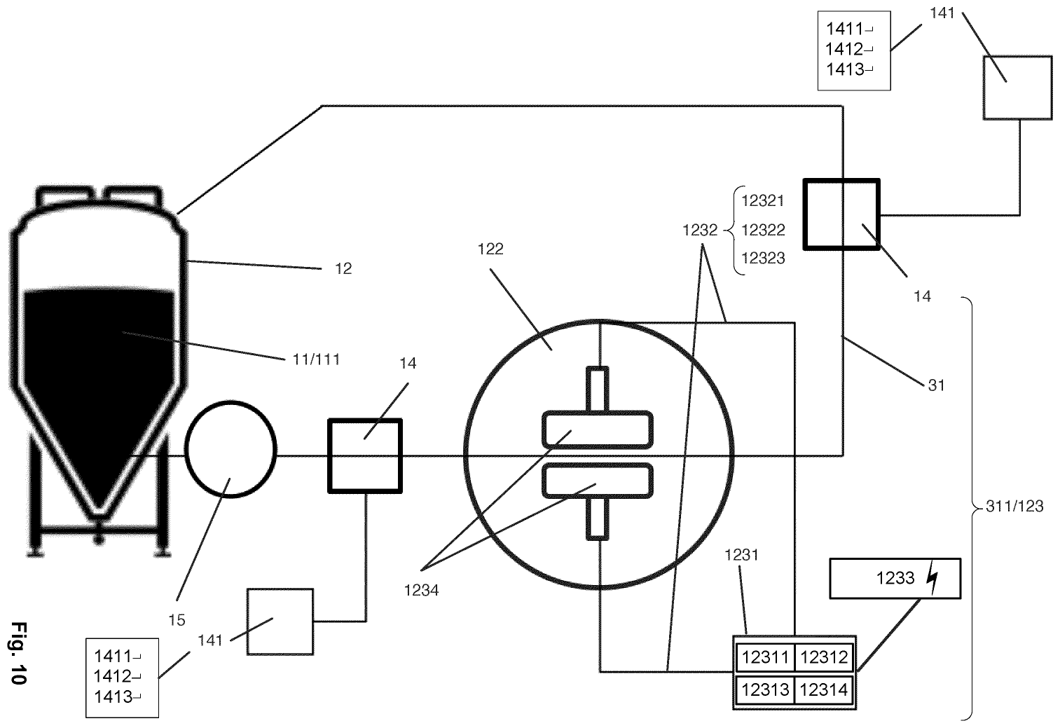

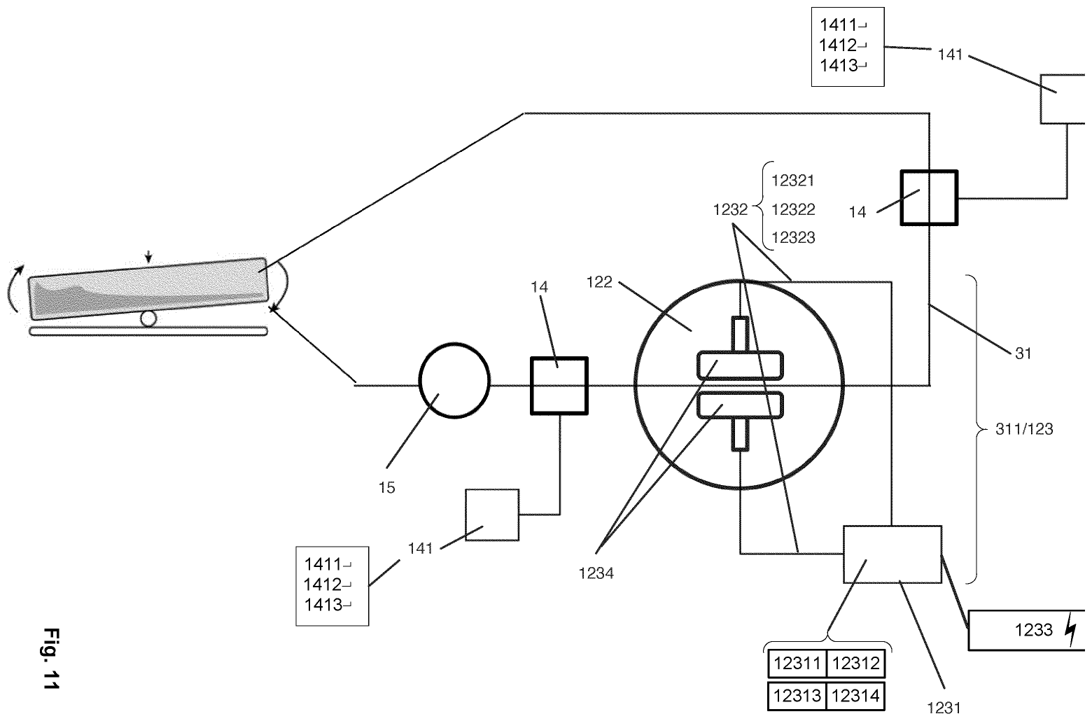

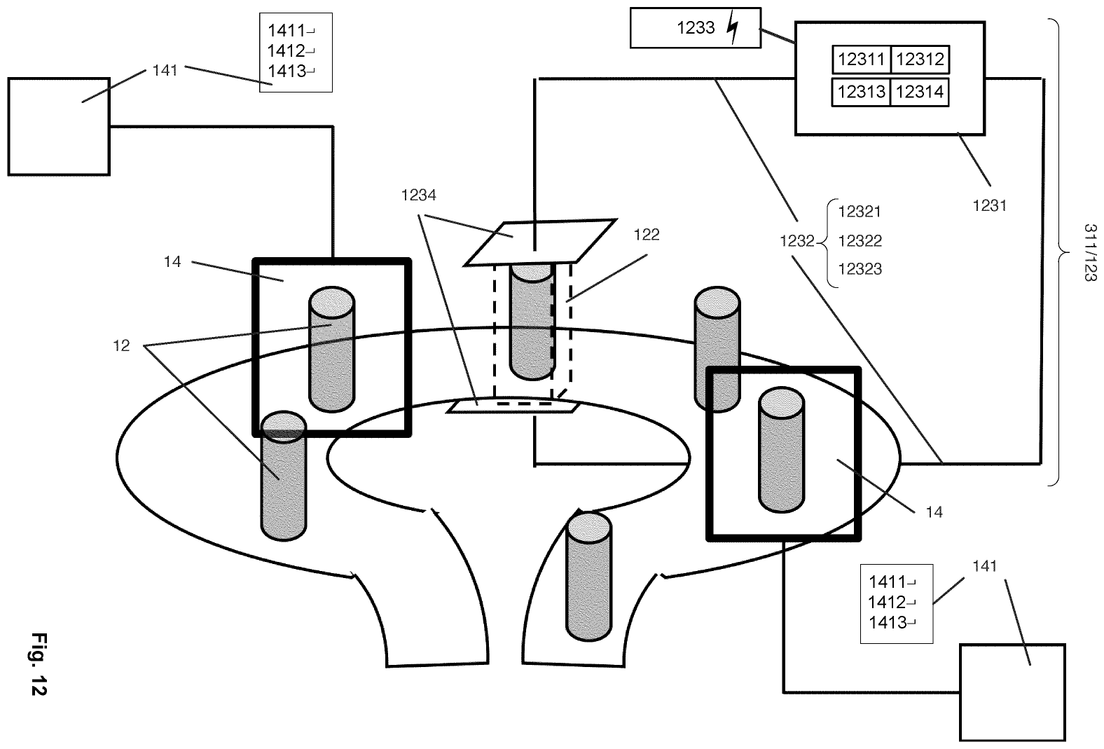

The above relation is limited to capturing spherical cells, and states that the induced membrane potential (Vₘ) is proportional to the radius of the cell (R) and the applied electric field (E), and will not be uniform along the cell membrane, as shown in FIG. 4. Extensions of this model have been developed to allow capturing more complex structures, such as irregular-shaped cells that can't be accurately described as spherical or ellipsoid. The Schwan relation (steady-state) allows capturing the effect of an applied electric field (E) on the transmembrane potential (Vₘ) given a cell radius of (R) as a function of the angle (θ). In FIG. 4, the induced membrane potential varies along the cell membrane. This example applies the Schwan parametrization to a spherical cell with a radius of 10 μm and an applied field intensity of 44 kV/cm. In prior art, there has been a shift in methodology toward capturing or parametrizing the biomolecular events occurring when an electric field interacts with a lipid bilayer. Molecular dynamics (MD) simulations have helped to model some of the events associated with electro-permeabilization and can be used to visualize the dynamics associated with membrane breakdown. Up to now, limitations in the computational power have restricted MD to a period less than 1 millisecond following pulse delivery; however, a multitude of effects have been reported over much longer time periods. These effects appear to be highly dependent on several factors such as; pulse duration, electric field strength, number of pulses, and frequency of pulses delivered.

FIG. 5 shows a diagram schematically illustrating the exemplary working principles of PEF/nsPEF based processing of cultivated cells and their respective effects.

FIG. 6 shows a diagram schematically illustrating treatment windows for selective inactivation, inactivation by the example of microbial flora and microalgae Chlorella vulgaris, continuous extraction of high value-added ingredients, and growth stimulation.

FIG. 7 shows a block diagram schematically illustrating an embodiment variant of the inventive industrial bioreactor 1 with a dual cycle-controlled optimization process providing an optimized cultivation process 21 for cell cultures 5/51, cell components 5/512 or metabolic products 5/52 of the cells in a nutrient medium 11. The dual cycle-controlled optimization process comprises a cultivation optimization cycle 2 optimizing cultivation performance 241 of the cultivation process 21 by adjusting operational first parameters 1311 of the bioreactor 1 identifying a biologically optimized window for cultivation and/or treatment, and a treatment optimization cycle 3 optimizing a treatment process 32 applied to the cell culture 51 during the cultivation process 21 within the optimized treatment window by applying operational second parameters 1312 of the bioreactor 1, wherein the cultivation optimization cycle 2 comprises measuring 22 first sensory parameters 222 by first sensory devices 221 capturing the cultivation performance 241 of the cultivation process 21, wherein the first sensory parameters 222 are transmitted to a first analyzer 23 reconciling between a target cultivation performance 242 and the measured cultivation performance 241 and if the target cultivation performance 242 is not met, the operational first parameters 1311 are adjusted and the measuring 22 and reconciliation 24 is reiterated. If the target cultivation performance 242 is met, the treatment optimization cycle 3 is triggered which comprises measuring 32 second sensory parameters 322 by second sensory devices 321 capturing the treatment performance 341 of the treatment process 31 by measuring the treatment induced deviation in the cultivation performance 241, wherein the second sensory parameters 322 are transmitted to a second analyzer 33 reconciling between a target treatment performance 342 and the measured treatment performance 341 and if the target treatment performance 342 is not met, the operational second parameters 1312 are adjusted and the measuring 32 and reconciliation 34 is reiterated, otherwise the dual cycle-controlled optimization process is completed.

FIGS. 8 and 9 show a diagram schematically illustrating measuring data of the industrial treatment cycle, where the physical and chemical characteristics of a population of cells is measured by flow cytometry (FC) measurements. The diagram line with the reference number 012 shows correctly treated cell samples, the diagram lines with the reference numbers 018 and 019 show control samples without applied treatment process, and the diagram lines with the reference numbers 010, 016 and 0112 show wrong treated cell samples, i.e. having an applied treatment with not the correct or optimized combination of amplitude, pulse length and number of pulses. In the flow cytometry measurement process, the samples containing the cells are suspended in a fluid and injected into the flow cytometer measuring device. The sample is focused to ideally flow one cell at a time through a laser beam, where the light scattered is characteristic to the cells and their components. Cells are labeled with fluorescent markers so light is absorbed and then emitted in a specific band of wavelengths. In the diagram, the blue vertical line marks the start of the treatment process in the bioreactor.

FIG. 10 shows a block diagram schematically illustrating an industrial liquid bioreactor 1, which type of bioreactor can be used for the present invention, where the cell cultures or micro-organisms are grown in submerged in liquid medium. The industrial liquid bioreactor 1 comprises devices as a pump 15, sensors or sensory devices 14, treatment device 311, and a reactor vessel 12 containing the liquid nutrient medium 11/111. Such industrial liquid or solid state bioreactors 1 are vessels that are designed to provide an effective environment for enzymes or whole cells/cell structures to transform biochemicals into products. Inactivation of cells or sterilization is one of the applications which can be carried out in the bioreactor such as in water treatment. Such industrial bioreactors can also be used for a great variety of other bioreactor applications, including applications for cell growth, enzyme production, biocatalysis, biosensors, food production, milk processing, extrusion, tissue engineering, algae production, protein synthesis, and anaerobic digestion.

FIG. 11 shows a block diagram schematically illustrating an industrial wave bioreactor 1 which type of bioreactor is also usable for the present invention. Liquid bioreactor are not suitable for all cultivation, since industrial cultivation technology for the culture of animal, plant and insect cells is dependent on the batch volume required. Though, devices such as spinner flasks, roller bottles, T-flasks, and similar systems can be used, these devices can typically only produce 1 to 2 L of culture per batch due to their inherently limited oxygen transfer capabilities. For example, spinner flasks, though very popular for suspension culture, as well for anchorage-dependent systems on microcarriers, are typically only useful for volumes less than 1 L. For larger volumes, it is usually necessary to use stirred-tank bioreactors that are modified to reduce shear forces. These bioreactors are complex and expensive devices, yet they often do not provide an ideal environment for cell growth due to high local fluid shear and the use of bubble aeration. For the industrial production of 1 to 100 L of cell culture, which is, for example, a typical volume required for protein characterization, inoculum propagation, and pilot-scale production, the adaption of stirred tank technology to cell culture is a futile exercise because this design has intrinsically high local shear rates making scale-up very difficult. Instead, it is critical to understand the special demands of cell culture to directly satisfy these needs. These demands include shear sensitivity, use of bubble-free aeration and pre-sterilized media, and small oxygen uptake rates. For industrial use it is further essential that any cultivation system is as simple as possible to operate. In this sense, many otherwise useful devices, such as fluidized bed bioreactors and hollowfiber systems, have turned out to be too complex to displace the spinner flask as the workhorse of cell cultivation. However, the use of wave bioreactor 1 in industrial application allows sometimes to overcome these problems. The wave bioreactor typically consists of a presterilized chamber 121, for example an inflatable, flexible plastic chamber, that is partially filled with media and inoculated with cells. The remainder of the chamber 121 is inflated with air. The air is continuously passed through the headspace during the cultivation. Mixing and mass transfer are achieved by rocking the chamber back and forth. This rocking motion generates waves at the liquid-air interface, greatly enhancing oxygen transfer. The wave motion also promotes bulk mixing, and off-bottom suspension of cells and particles. This concept of using rocking for agitation can e.g. be used for the agitation of liquids in industrial assay plates and gels. The rocking device shown in FIG. 9 can e.g. consist of a platform that rotates in one axis through an angle that can be adjusted, for example, between 5° to 10°. Pneumatic bellows can be used to rock the platform, for example, at a rocking rate that can be adjusted from 5 to 40 rocks/minute (rpm). If a pneumatic system is used, this allows to ensure that unit does not generate any heat during operation, enabling it to be placed inside an incubator. A typically pre-sterilized disposable cell culture chamber 121 is placed on the platform which is partially filled with media 111 and then inflated using the integral sterile inlet filter. Air can be continuously passed through the headspace of the chamber 121. This airflow provides oxygenation 141122 and gas exchange for pH control 141121 and CO₂ removal. Exhaust air passes through a sterilizing filter and a backpressure control valve. A backpressure control valve can ensure that the chamber 121 is always fully inflated at any airflow. Such a valve can also prevent overinflation and potential bursting of the chamber 121. Temperature 14111 and pH control 141121 can e.g. be achieved by placing the entire unit inside a conventional cell culture CO₂ incubator. Alternatively, temperature control 14111 can e.g. be achieved by heating the underside of the culture chamber 121. The wave motion promotes good bulk liquid 111 movement and minimizes the occurrence of temperature gradients. The industrial wave bioreactor 1 further comprises devices as a pump 15, sensors or sensory devices 14, treatment device 311, and as reactor vessel 12 the chamber 121 containing the liquid nutrient medium 11/111.

FIG. 12 shows a block diagram schematically illustrating a solid-state bioreactor which type of bioreactor is also usable for the present invention. Solid-state bioreactors (SSF: Solid-State Fermentation) have, inter alia, important advantages over the other type of bioreactors with submerged fermentation, such as lower water use and higher volumetric productivity. As a result, SSF technology covers an increasing field in the industrial application of bioreactors in the past few decades. SSF bioreactors can be based on various types of reactor technology, as for example tray, packed-bed, rotating/stirred-drum, fluidized-bed, rocking-drum, and stirred-aerated bioreactors. Technically important is typically the reactor design, the control of heat and mass transfer, and the applied operational strategies. Both, industrial cultivation of cells as well as the growth of microorganisms can be realized in SSF bioreactors. Important for the industrial applications of SSF bioreactors are, as mentioned, typically the control of heat transfer on a large scale and to quantitative characterization and measurement of the cell growth. For the latter, one of the technical challenges are e.g. the appropriate selection of nondestructive detection methods during the SSF processes. In general, compared to submerged fermentation, the solid media 112 used in SSF contain less water, however, an important gas phase typically exists between the particles. This feature is of great technical importance because of the poor thermal conductivity of the air compared to the water. Another point is the wide variety of matrices 112 used in SSF bioreactors which vary in terms of composition, size, mechanical resistance, porosity and water holding capacity. All these factors can affect the reactor design and the control strategy for the parameters. Indeed in submerged fermentation, all the media can be considered to be made up essentially of water. Thus, in liquid bioreactors, the temperature 14111 and pH 141121 regulations are typically simple to achieve and pose no problem during the industrial scaling-up of a process. In submerged fermentation, the major difficulty is typically the transfer of oxygen to micro-organisms or cells which depends upon the shape, the size of the reactor and the agitation/aeration system used. This transfer can be measured by a parameter KLa (oxygen transfer coefficient) where its value expresses the capacity of the equipment to transfer oxygen independently of the volume of the reactor and so, constitutes an important parameter used for the scale-up design in submerged fermentation. In contrast to liquid bioreactors, in SSF bioreactors, besides the oxygen transfer 141122 which can be a limiting factor for some designs, the technical problems are more complex affecting the control of two important parameters, namely the temperature 14111 and the water content of the solid medium 112, i.e. the substrate 112. Concerning the various types of solid-state bioreactors, it is important to note that generally many types of reactors are able to run a cultivation process at laboratory-scale with small quantities of medium 112. But the industrial scale-up is often complicated inter alia due to intense heat generation and heterogeneity in the system, which also complicates the technical control of the cultivation process. In industrial solid-state bioreactor, the above-discussed heat and mass transfer problems can, inter alia, attribute to poor aeration. This problem can e.g. be addressed by (i) circulating the air around the substrate layer 112 or (ii) leading the air through the substrate layer 112. For the latter, three possibilities are used in the prior art, which are unmixed, intermittently or continuously mixed beds. However, as already mentioned, the present invention can also be used with solid-state bioreactors. As shown in FIG. 12, a suitable industrial solid-state bioreactor 1 can e.g. be realized comprising a pump 15, sensors or sensory devices 14, treatment device 311, and as reactor vessel 12 the cultivation containers 121 containing the substrate 11/112.

### DETAILED DESCRIPTION OF THE INVENTION

FIG. 7 illustrates, schematically, an architecture for a possible implementation of an embodiment of the inventive industrial bioreactor 1 with a dual cycle-controlled optimization process providing an optimized cultivation process 21 for cell cultures 5/51, cell components 5/512 or metabolic products 5/52 of the cells in a nutrient medium 11. The industrial bioreactor 1 comprises a reactor vessel 12 providing controlled bioreactor conditions for the cultivation process 21. The bioreactor is an industrial bioreactor for the production of cells or sell cultures, such as cultured meat production. The bioreactor 1 can e.g. also be realized as a fermenter and/or a germination box in case of seed germination applications. Further, the bioreactor can also be an industrial bioreactor for the production of metabolic products, such as biologically active secondary metabolites (antibiotics, bacterial toxins, immune drugs, and alkaloids), single cell proteins, enzymes, industrial chemicals, biofuel, food, phenolics, feed, and pharmaceutical products. However, it is to be noted, that the present invention can be applied to any kind of liquid bioreactors or solid-state bioreactor, such as Solid-State Fermentation (SSF) bioreactors or Submerged Fermentation (SmF) bioreactors, inter alia, used in the field of waste management technology, and applications including bioremediation, detoxification, bioleaching and biopulping etc. In a preferred embodiment variant, the bioreactor is used in food production such as cultured meat or fermented food, e.g. tempeh, miso, koji, and soy sauce etc. The bioreactor 1 can e.g. comprise one or a plurality of substrate container 121 (also referred as trays) enterable into the reactor vessel 12, wherein the cell culture 51 is cultivated on a nutrient medium 11 also contained in the substrate container 121. However, the bioreactor can not only be realized as a tray bioreactor, but also e.g. as a packed bed bioreactor, an air pressure pulsation bioreactor, or an intermittent or continuously mixed SSF bioreactors etc. If the bioreactor is used for cultured meat production may comprise a scaffold holding the one or more substrate container 121 comprising a population of cells. The cultured meat product produced in the bioreactor can e.g. have a thickness from about 100 μm to about 500 mm, or any other size. In an embodiment, a method of producing such a cultured meat product may comprise preparing the scaffold, placing the scaffold into a bioreactor, adding the substrate container 121 with the nutrient medium 11 and the cell culture or population of cells to the bioreactor 1, culturing and cultivating, respectively, the population of cells in the bioreactor containing the scaffold for a period of time, hereby forming the cultured meat product, and removing the cultured meat product from the bioreactor 1. During the cultivation process 21 in the bioreactor 1, a treatment 31 can be applied to the cell cultures, as described below. The cultured meat product can e.g. be configured to mimic the taste, texture, size, shape, and/or topography of a traditional slaughtered meat. As used herein, the term “traditional slaughtered meat” means one or more types of meat obtained from a once-living animal for the purpose of consumption. Such meat is generally, although not always, obtained from livestock, fish, or other animals raised or slaughtered primarily for food production purposes. Non-limiting examples of traditional slaughtered meat include chicken, turkey, pork, beef, fish, and the like. Traditional slaughtered meat is generally appropriate for consumption by one or more mammal species. Further as used herein, the term “cultured meat product” means a meat product that is produced by human or machine intervention, rather than grown as a natural component of a living animal. A cultured meat product is thus not obtained directly from the slaughter of a living animal, but rather by an artificial cultivation process e.g. induced in a controlled bioreactor environment. Like traditional slaughtered meat, a cultured meat product is generally appropriate for consumption by one or more mammal species.

A control unit 13 of the bioreactor 1 is connected to sensory devices 14 measuring sensory parameter 141 values comprising at least measures related to the composition 1411 of the nutrient medium 11 and/or concentration 1412 of the nutrient medium 11 and/or oxygen 1413 and/or temperature 1414 and/or pH-value 1415 and/or sterility 1416 and transmitting them to the control unit 13. The cultivation process 21 is controlled and/or steered by the control unit 13 by adjusting operational parameters of the bioreactor 1 affecting the measured sensory parameter 141 values.

The dual cycle-controlled optimization process comprises a cultivation optimization cycle 2 optimizing cultivation performance 241 of the cultivation process 21 by adjusting operational first parameters 1311 of the bioreactor 1 identifying a biologically optimized window for cultivation and/or treatment. Further, the dual cycle-controlled optimization process comprises a treatment optimization cycle 3 optimizing a treatment process 32 applied to the cell culture 51 during the cultivation process 21 within the optimized treatment window by applying operational second parameters 1312 of the bioreactor 1.

The cultivation optimization cycle 2 comprises measuring 22 first sensory parameters 222 by first sensory devices 221 of the sensory devices 14 capturing the cultivation performance 241 of the cultivation process 21. The first sensory parameters 222 are transmitted to a first analyzer 23 reconciling between a target cultivation performance 242 and the measured cultivation performance 241 and if the target cultivation performance 242 is not met, the operational first parameters 1311 are adjusted and the measuring 22 and reconciliation 24 is reiterated. The first sensory devices 221 measuring the cultivation performance 241 of the cultivation process 21 by means of the first sensory parameters 222 can e.g. comprise a measuring device 2211 for bioimpedance measurement detecting their response to electric excitation, where by means of electrodes a current- or potential-based excitation signal is applied to the cell culture and the response is measured converting the charge to ionic charge and vice versa providing detection of at least cell number 2221 and/or cell size 2222 and/or cell viability 2223 of the cells 51.

If the target cultivation performance 242 is met, the treatment optimization cycle 3 is triggered which comprises measuring 32 second sensory parameters 322 by second sensory devices 321 of the sensory devices 14 capturing the treatment performance 341 of the treatment process 31 by measuring the treatment induced deviation in the cultivation performance 241. The second sensory parameters 322 are transmitted to a second analyzer 33 reconciling between a target treatment performance 342 and the measured treatment performance 341 and if the target treatment performance 342 is not met, the operational second parameters 1312 are adjusted and the measuring 32 and reconciliation 34 is reiterated, otherwise the dual cycle-controlled optimization process is completed.

For example, the treatment process 31 can e.g. comprise applying nanosecond pulsed electric fields (nsPEF) 3113 to the cell culture using at least two applied electrodes 3112, the electric fields 3113 being applied by coupling one electrode 3112 to higher voltage and one electrode 3112 to ground or lower voltage, and the pulsed electric fields having a definable shape 31131 and/or frequency 31132 and/or strength 31133. As a variant, the control unit 13 can e.g. comprise predefined basic nanosecond pulsed electric fields 3113 settings for each possible cell type 511.

The nanosecond pulsed electric fields (nsPEF) 3113 can e.g. be applied by electrodes to the bioreactor 1, as such. If the bioreactor comprises one or more substrate container 121 holding the nutrient medium 11 and the cell culture 51, the electrodes can also be applied to each substrate container 121, e.g. by integrating them into possible walls of the substrate container 121. Finally, the bioreactor 1 can also include a pulsed electric field (PEF) station or be included in a pulsed electric field (PEF) station providing the required pulsed electric fields. In the latter cases, the biological materials can e.g. flow into the PEF station via an inlet, may be treated, and may then be released via an outlet. The PEF generation can e.g. be provided by a pulse generator, where the PEF treatment is effective in a particular treatment zone comprising the cell culture 51. In particular, materials contained in or passing through the treatment zone are subjected to non-arcing electric field pulses generated by the pulse generator. The electric field pulses can e.g. be generated by applying a voltage pulse to the electrodes, the pulse can e.g. have a square-wave shape. However, the pulses may also have an exponentially decaying or oscillatory shape. Further, the pulses may be monopolar, bipolar, or even instant reverse charges. The electric field pulses can e.g. be of a preferred duration of 10 to several 100 nanoseconds, however dependent on the treatment, the pulse duration can e.g. lie also in the range of 2 to 15 microseconds with a peak field strength of 15 to 100 kV/cm etc. The resulting duration of treatment can e.g. be a function of the shape of the treatment zone (e.g., electrodes) and the characteristics of the electric field pulses. Preferable, the treatment is applied during a larger part or the whole cultivation process 21. The pulse generator can e.g. be coupled to a power supply, which the pulse generator uses to generate a series of high voltage non-arcing electric field pulses across electrodes associated with the treatment zone. Depending on the power supply used, a voltage transformer can e.g. be included, coupled between the power supply and the pulse generator. The pulse generator can include a bank of capacitors and switching circuitry that may connect the bank of capacitors across the electrodes to create the pulses within the treatment chamber. A switching circuitry can e.g. be controlled by a controller that has as an input a signal from a signal generator. By varying the characteristics of the signal from the signal generator, the characteristics of the pulses in the treatment zone can be varied.

As an embodiment variant, in the bioreactor vessel 12 or the substrate container 121 can be disposed electrodes, one of the electrodes coupled to a higher voltage and the other the electrodes coupled to ground or a lower voltage. Insulators can be disposed at either side of the electrodes and between the electrodes. The insulators, as well as the substrate container 121, which can e.g. be made of an insulating material, isolate the electrodes from couplings which may be attached or secured to either end of the substrate container 121. Similarly, the insulator and the substrate container 121 space the electrodes to define the treatment zone disposed therebetween. In operation, the biological materials, i.e. the cell culture 51, are to be treated in the treatment zone e.g. during the cultivation process 21.

The treatment performance 341 can e.g. be measured by means of dielectric spectroscopy 3211 measuring the dielectric properties of the cells 51 as a function of frequency, wherein the frequency-dependent permittivity in a target range is measured, and wherein the measured amplitude or signal intensity serving as a measured target parameter value for the performance of the treatment. The target range can e.g. lie at 0.1-30 MHz. Thus, the treatment performance 341 can e.g. measured by means of a flow cytometer 3212 as at least one of the second sensory devices 321 measuring metabolic activity based on a fluorescence assay, a conversion of the fluorescent dye and the signal intensity serving as a measured target parameter value for the performance of the treatment. The fluorescence assay can e.g. be fluorescein diacetate (FDA). The flow cytometer 3212 can e.g. comprise at least a measuring system and a detector and an amplification unit, the flow cytometer being connected to and transferring measuring signals to the control unit 13 for analysis of the transmitted signals. The measuring system measures quantities such as impedance or conductivity can e.g. use optical systems realized at the bioreactor 1, the optical systems emitting light signals. The detector can e.g. comprise an analog-to-digital conversion ADC system converts analog measurements of forward-scattered light (FSC), side-scattered light (SSC), and dye-specific fluorescence signals into digital signals being processable by the control unit 13. The amplification unit can e.g. be linear or logarithmic realized.

Thus, the industrial bioreactor with a dual cycle-controlled optimization process provides a reliable and controllable detection of optimal processing parameters for an industrial cultivation process in a bioreactor by detecting in a first step the optimal biological cultivation window based on the cell characteristics of the cells to be cultivated. In a second step, the technical treatment is optimized, i.e. the optimal treatment window for the operational parameters of the treatment to be applied, is automatically detected. This first step can continue during the whole treatment and, thus, can serve to identify the treatment stop while the second step allows to quantify the process efficiency and thus detects the percentage increase of the cultivation/growth efficiency and thus further also allows the prediction of the process runtime. Therefore, a phase angle 2224 of the cell culture 51 can e.g. be measured by the bioimpedance measurement 22, wherein the phase angle 2224 is correlated with the cell viability, and wherein with increasing measured phase angle 2224 the treatment 31 is applied while with decreasing measured phase angle 2224, the treatment 31 is stopped. The treatment performance can e.g. be detected or measured to be optimized, if an acceleration of the cultivation process 21 and/or a targeted biological growth is measured in the bioreactor 1 based on the measured second sensory parameter values 322.

Finally, as a further suitable embodiment 4 variant, the control unit 13 can e.g. comprise a machine-learning or artificial-intelligence based unit 132 capturing the measured treatment performance 341 measured by means of dielectric spectroscopy 3211 and/or the measured treatment performance 341 measured by means of the flow cytometer 3212. In this embodiment variant, the operational first parameters 1311 and/or operational second parameters 1312 are automatically adapted by the control unit 13, wherein at least the first and secondary sensory parameter 222/322 are applied as input values to the machine-learning or artificial-intelligence based unit 132, and wherein the output of the machine-learning or artificial-intelligence based unit 132 triggers the adjustment of the operational first parameters 1311 and/or operational second parameters 1312 until the target cultivation performance 242 with the applied treatment process 32 is reached. As already discussed above, the machine-learning module can e.g. be based on supervised or unsupervised learning structures. As an example, machine-learning module can e.g. convert the measured first and/or second sensory parameters into a sequence of assigned binary input codes and process the sequence of the assigned binary input codes by applying a maximum likelihood parameter estimation for the training of a multi-dimensional data structure of the machine-learning module with the variable hidden Markov model parameters, wherein the elements of the sequence of storable parameter states of the Markov chain are assumed to be independent measurements of each other and wherein the model parameters of the multi-dimensional data structure are varied by maximizing the multiplied product of the probabilities in order to obtain the trained model parameters of the multi-dimensional data structure. The model parameters of the multi-dimensional data structure can e.g. be iteratively varied until a predefined convergence threshold is triggered. For determining said threshold value of a score indicating or providing a measure for the optimization of the operational first and/or second operational parameters, an averaging process can e.g. be applied based on the different pattern of first and/or second sensory parameters of the sensory and/or measuring data of an identified time frames. In an embodiment variant, the sensitivity of the chosen operational parameters can e.g. be automatically tuned based on dynamic adjustments of the threshold value. This embodiment variant has inter alia the advantage, that the convergence speed by training the variable hidden Markov model parameters of the multi-dimensional data structure can be optimized. The embodiment variant has, inter alia, the advantage that it provides a novel method and bioreactor for automated detection, measuring and triggering of an optimized setting for the first and second operational parameters affecting the cultivation of the cell cultures and the applied treatment used in industrial bioreactor. It provides an efficient automated system for controlling and monitoring small- to large-scale industrial bioreactors (e.g. used for cultured meat production), which typically are difficult to handle. In addition, this embodiment variant has, inter alia, the advantage that the AI integration allows to serve finally to treat also completely unknown organisms without evaluating appropriate settings for the first and/or second operational parameters of the bioreactor empirically, since the system optimizes itself by means of the actual/target comparison.

### LIST OF REFERENCES

1 Industrial bioreactor
   11 Nutrient Medium
      111 Liquid medium
      112 Substrate
   12 Reactor vessel
      121 Substrate Container
      122 Treatment Zone/Treatment Chamber
      123 Pulsed Electric Field Unit
         1231 Pulse Generator
         12311 Signal Generator
         12312 Controller
         12313 Capacitors
         12314 Switching circuit
         1232 Electric Pulse
         12321 Electric Pulse Shape
         12322 Electric Pulse Frequency
         12323 Electric Field Strength
         1233 Power Supply
         1234 Electrodes
   13 Control unit
      131 Operational parameters
         1311 Operational first parameters
         1312 Operational second parameters
      132 Machine-learning or artificial-intelligence based unit
   14 Sensory devices/Sensors
      141 Sensory parameters
         1411 Physical sensory parameters
         14111 Temperature
         1412 Chemical sensory parameters
         141121 pH-Value
         141122 Oxygen
         141123 Composition of the nutrient medium
         141124 Concentration of the nutrient medium
         1413 Biological sensory parameters
         141131 Cell concentration
         1451132 Sterility (e.g. decimal reduction time D-value)
   15 Pump
2 Cultivation Optimization Cycle
   21 Cultivation Process
   22 Measuring of First Sensory Data
      221 First Sensory Devices
         2211 Bioimpedance measurement device
      222 First Sensory Parameters
         2221 Cell Number
         2222 Cell Size
         2223 Cell Viability
         2224 Phase angle
   23 Analyzer
   24 Cultivation Performance Target/Actual Reconciliation
      241 Actual Cultivation Performance
      242 Target Cultivation Performance
   25 Threshold/Trigger Not Reached
   26 Threshold/Trigger Reached
3 Treatment Optimization Cycle
   31 Treatment Process
      311 Treatment device
         3111 Bio-based PEF Treatment Device
         3112 Electrodes
         3113 Pulsed Electric Field (PEF)
         31131 Pulse shape
         31132 Pulse frequency
         31133 Field strength
   32 Measuring of Second Sensory Data
      321 Second sensory devices
         3211 Dielectric spectroscopy system
         3212 Flow cytometer
      322 Second Sensory Parameters
   33 Analyzer
   34 Second Target/Actual Reconciliation (target/performance analysis)
   35 Threshold/Trigger Not Reached
   36 Threshold/Trigger Reached
4 Stop Process
5 Biomass
   51 Cells/Cell Culture
      511 Cell Type
      512 Components of Cells
   52 Metabolic Products of the Cell Culture

1.-16. (canceled)

17. An industrial bioreactor with a cycle-controlled process providing a cultivation process for cell cultures in a nutrient medium, the industrial bioreactor comprising:
a reactor vessel providing controlled bioreactor conditions for the cultivation process, and
control circuitry connected to sensory devices measuring sensory parameter values comprising at least measures related to the nutrient medium and transmitting them to the control circuitry, wherein the control circuitry controls and steers the cultivation process of the bioreactor,
wherein:
a dual cycle-controlled process of the control circuitry comprises a cultivation cycle and a treatment cycle, wherein, for the cultivation cycle, the sensory devices comprise first sensory devices measuring the cultivation performance of the cultivation process, the first sensory devices comprising a measuring device for electrical measurement of bioimpedance detecting their response to electric excitation, where by means of electrodes a current- or potential-based excitation signal is applied to the cell culture and the response is measured converting the charge to ionic charge and vice versa providing detection of at least cell number and/or cell size and/or cell viability of the cells, wherein the cultivation process is completed by the control circuitry, if a target cultivation performance is met, and
the control circuitry comprises a trigger triggering the treatment cycle, if a target cultivation performance is met, the sensory devices comprise second sensory devices capturing the treatment performance of the treatment process by measuring the treatment induced deviation in the cultivation performance, wherein the dual cycle-controlled process is completed by the control circuitry, if a target treatment performance is met.

18. The industrial bioreactor with a dual cycle-controlled process according to claim 17, wherein:
the measuring sensory parameter values comprise at least measures related to the composition of the nutrient medium and/or concentration of the nutrient medium and/or oxygen and/or temperature and/or pH-value and/or sterility.

19. The industrial bioreactor with a dual cycle-controlled process according to claim 17, wherein:
alternatively a phase angle of the cell culture is measured by the bioimpedance measurement, the phase angle being correlated with a cell viability, wherein with increasing measured phase angle the treatment is applied while with decreasing measured phase angle, the treatment is stopped.

20. The industrial bioreactor with a dual cycle-controlled process according to claim 17, wherein:
the treatment process comprises applying nanosecond pulsed electric fields to the cell culture using at least two applied electrodes, the electric fields being applied by coupling one electrode to higher voltage and one electrode to ground or lower voltage, and the pulsed electric fields having a definable shape and/or frequency and/or strength.

21. The industrial bioreactor with a dual cycle-controlled process according to claim 20, wherein:
the control circuitry comprises predefined basic nanosecond pulsed electric fields settings for each possible cell type.

22. The industrial bioreactor with a dual cycle-controlled process according to claim 17, wherein:
the treatment performance is measured by means of dielectric spectroscopy system measuring dielectric properties of the cells as a function of frequency, wherein the frequency-dependent permittivity in a target range of the frequency is measured, and wherein the measured amplitude or signal intensity serving as a measured target parameter value for the performance of the treatment.

23. The industrial bioreactor with a dual cycle-controlled process according to claim 22, wherein:
the target range having 0.1-30 MHz.

24. The industrial bioreactor with a dual cycle-controlled process according to claim 20, wherein:
the treatment performance is measured by means of a flow cytometer as at least one of the second sensory devices measuring metabolic activity based on a fluorescence assay, a conversion of a fluorescent dye and a signal intensity serving as a measured target parameter value for the performance of the treatment.

25. The industrial bioreactor with a dual cycle-controlled process according to claim 24, wherein:
the fluorescence assay is fluorescein diacetate.

26. The industrial bioreactor with a dual cycle-controlled process according to claim 24, wherein:
the flow cytometer comprises at least a measuring system and a detector and an amplifier, the flow cytometer being connected to and transferring measuring signals to the control circuitry for analysis of the transmitted signals.

27. The industrial bioreactor with a dual cycle-controlled process according to claim 26, wherein:
the measuring system measures impedance and/or conductivity and/or pH using optical systems emitting light signals.

28. The industrial bioreactor with a dual cycle-controlled process according to claim 26, wherein:
the detector comprises an analog-to-digital conversion system converts analog measurements of forward-scattered light, side-scattered light, and dye-specific fluorescence signals into digital signals being processable by the control circuitry.

29. The industrial bioreactor with a dual cycle-controlled process according to claim 26, wherein:
the amplifier includes a linear or logarithmic amplifier.

30. The industrial bioreactor with a dual cycle-controlled process according to claim 17, wherein:
the treatment performance is optimized if an acceleration of the cultivation process and/or a targeted biological growth is measured in the bioreactor based on the measured second sensory parameter values.

31. The industrial bioreactor with a dual cycle-controlled process according to claim 17, wherein:
the bioreactor is realized as a fermenter and/or a germination box in case of seed germination applications.

32. The industrial bioreactor with a dual cycle-controlled process according to claim 17 wherein:
the control circuitry comprises a machine-learning or artificial-intelligence based circuitry capturing the measured treatment performance measured by means of dielectric spectroscopy system and/or the measured treatment performance measured by means of the flow cytometer, wherein the operational first parameters and/or operational second parameters are automatically adapted by the control circuitry, wherein at least the first and secondary sensory parameter are applied as input values to the machine-learning or artificial-intelligence based circuitry, and wherein the output of the machine-learning or artificial-intelligence based circuitry triggers the adjustment of the operational first parameters and/or operational second parameters until the target cultivation performance with the applied treatment process is reached.
